(19) United States
(12) Patent Application Publication
(10) Pub. No.: US 2010/0173910 A1
(43) Pub. Date: Jul. 8, 2010

(54) TRIAMINOPYRIMIDINE CYCLOBUTENEDIONE DERIVATIVES USED AS PHOSPHATASE CDC25 INHIBITORS

(75) Inventors: Anne-Marie Liberatore, Auffargis (FR); Dominique Pons, Paris (FR); Dennis Bigg, Gif Sur Yvette (FR); Grégorie Prevost, Antony (FR); Marie-Christine Brezak Pannetier, Antony (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: IPDRN PHSTMS D.S.S., BOULOGNE-BILLANCOURT (FR)

(21) Appl. No.: 12/668,999

(22) PCT Filed: Jul. 10, 2008
(86) PCT No.: PCT/FR08/01006

§ 371 Date: Mar. 2, 2010

(30) Foreign Application Priority Data
Jul. 13, 2007 (FR) 0705094

(51) Int. Cl. A61K 31/5377; A61P 35/00; C07D 403/12; A61K 31/506; C07D 413/12

(52) U.S. Cl. 514/235.8; 544/323; 514/275; 544/122

(57) ABSTRACT

The present invention relates to triaminopyrimidine derivatives of formula (I)

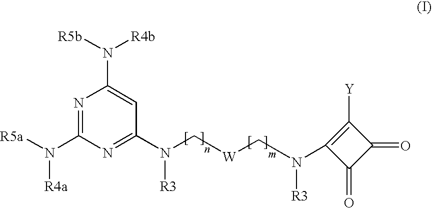

where Y, R3, W, R4a, R5a, R4b, R5b, n and m are variable. These compounds have CDC25 phosphatase-inhibiting activity and can therefore be used as drugs in diseases in which CDC25 phosphatases are involved. The invention also relates to pharmaceutical compositions containing said products and methods of using the drug.

The present invention relates to novel triaminopyrimidine derivatives. These compounds have CDC25 phosphatase-inhibiting activity and can therefore be used as drugs in diseases in which CDC25 phosphatases are involved. The invention also relates to pharmaceutical compositions containing said products and to the use thereof to prepare a drug.

The transition between the different phases of the cell cycle during mitosis or meiosis is controlled by a group of proteins whose enzymatic activity is associated with different phosphorylation states. These states are controlled by two large classes of enzyme: kinases and phosphatases.

In this way, the synchronisation of the different phases of the cell cycle enables the cell structure to be reorganised at each cycle in all living organisms (microorganisms, yeasts, vertebrates, plants). One group of kinases, the cyclin-dependent kinases (CDKs), has a major role in cell cycle control. The enzymatic activity of these various CDKs is controlled by two other families of enzymes that work in opposition (Jessus and Ozon, Prog. Cell Cycle Res. (1995), 1, 215-228). The first comprises kinases such as Wee1 and Mik1, which deactivate CDKs by phosphorylating certain amino acids (Den Haese et al., Mol. Biol. Cell (1995), 6, 371-385). The second comprises phosphatases such as CDC25, which activate CDKs by dephosphorylating tyrosine and threonine residues of CDKs (Gould et al., Science (1990), 250, 1573-1576).

Phosphatases are classified into 3 groups: the serine/threonine phosphatases (PPases), the tyrosine phosphatases (PTPases) and the dual-specificity phosphatases (DSPases). These phosphatases play an important role in the regulation of many cell functions.

As far as the human CDC25 phosphatases are concerned, 3 genes (CDC25-A, CDC25-B and CDC25-C) encode the CDC25 proteins. In addition, variants originating from alternative splicing of the CDC25B gene have been identified: these splice variants are CDC25B1, CDC25B2 and CDC25B3 (Baldin et al., Oncogene (1997), 14, 2485-2495).

The role of CDC25 phosphatases in oncogenesis is now better understood and the mechanisms through which these phosphatases act are illustrated in the following references in particular: Galaktionov et al., Science (1995), 269, 1575-1577; Galaktionov et al., Nature (1996), 382, 511-517; and Mailand et al., Science (2000), 288, 1425-1429.

Overexpression of the various forms of CDC25 has now been reported in many human tumour series, for example:
Breast cancer: see Cangi et al., Abstract 2984, AACR meeting San Francisco, 2000);
Lymphoma: see Hernandez et al., Int. J. Cancer (2000), 89, 148-152 and Hernandez et al., Cancer Res. (1998), 58, 1762-1767;
Head and neck cancers: see Gasparotto et al., Cancer Res. (1997), 57, 2366-2368;
Pancreatic cancers: see Junchao Guo et al., Oncogene (2004), 23, 71-81.

Moreover, E. Sausville's group has reported a negative correlation between the level of CDC25-B expression in a panel of 60 cell lines and their sensitivity to CDK inhibitors, suggesting that the presence of CDC25 can provide resistance to certain antitumour agents and more particularly to CDK inhibitors (Hose et al., Proceedings of AACR, Abstract 3571, San Francisco, 2000).

In addition to other targets, compounds capable of inhibiting CDC25 phosphatases are therefore being sought at present, particularly with a view to using them as anticancer agents.

CDC25 phosphatases also have a role in neurodegenerative diseases (see Zhou et al., Cell Mol. Life Sci. (1999), 56(9-10), 788-806; Ding et al., Am. J. Pathol. (2000), 157(6), 1983-90; Vincent et al., Neuroscience (2001), 105(3), 639-50) and the use of compounds with inhibitory activity against these phosphatases can therefore also be envisaged in the treatment of these diseases.

In addition, CDC25 phosphatases have a role in disorders/diseases such as organ transplant rejection and certain autoimmune diseases. These disorders/diseases involve inappropriate activation of lymphocytes and monocytes/macrophages. Current immunosuppressant drugs have side effects that could be alleviated or modified by products that specifically target the signalling pathways in hemopoietic cells that initiate and maintain inflammation, and therefore the use of compounds that inhibit these phosphatases can also be envisaged for the treatment of these diseases.

The Applicant has discovered that triaminopyrimidine derivatives having the general formula (I) described below have CDC25 phosphatase-inhibiting activity. In view of the above, these compounds could be used as drugs, particularly in the treatment and/or prevention of the following diseases or disorders:
inhibition of tumour proliferation, alone or in combination with other treatments;
cancers;
inhibition of the proliferation of normal cells, alone or in combination with other treatments;
neurodegenerative diseases;
prevention of spontaneous alopecia;
prevention of alopecia induced by exogenous products;
prevention of radiation-induced alopecia;
prevention of spontaneous or induced apoptosis of normal cells;
prevention of meiosis and/or fertilisation;
prevention of oocyte maturation;
autoimmune diseases;
graft rejection;
allergies;
any diseases/disorders corresponding to uses reported for CDK inhibitors, and particularly noncancerous proliferative diseases (for example, angiogenesis, psoriasis or restenosis), cancerous proliferative diseases, parasitic diseases (protozoan proliferation), viral infections, neurodegenerative diseases, myopathies; and/or
any diseases/disorders corresponding to clinical applications of vitamin K and its derivatives.

In addition, on account of their CDC25 phosphatase-inhibiting properties, the compounds of the present invention could also be used to inhibit or prevent the proliferation of microorganisms, and particularly yeasts.

Thus, the invention firstly relates to a compound having the general formula (I)

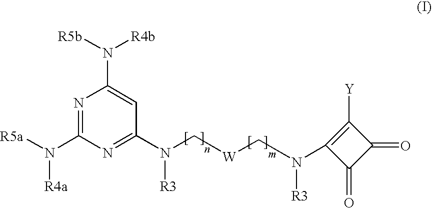

in a racemic form, an enantiomeric form or any combination thereof, where:
Y represents independently an NR1R2 or OR13 group;
W represents independently —NR6- or —CR6R7-;
R3 represents a hydrogen atom or an alkyl group;
n and m are integers from 0 to 4 inclusive;
R4a and R5a represent independently a hydrogen atom, an alkyl group or alternatively together with the nitrogen atom to which they are attached form a heterocycloalkyl;
R4b and R5b represent independently a hydrogen atom, an alkyl group or alternatively together with the nitrogen atom to which they are attached form a heterocycloalkyl;
R1, R2 and R13 represent independently a hydrogen atom or a group selected from:
alkyl,
arylalkyl optionally substituted by one or more identical or different halo groups;
heteroaryl optionally substituted by one or more identical or different groups selected from: halo, hydroxy, cyano, nitro, alkyl, alkoxy, haloalkyl, haloalkoxy;
aryl optionally substituted by one or more identical or different groups selected from: halo, hydroxy, cyano, nitro, alkyl, alkoxy, haloalkyl, haloalkoxy, aminoalkyl, alkylaminoalkyl, dialkylaminoalkyl, cycloalkyl, aryl, heteroaryl, —NH—(C═O)—R14, —(C═O)—R14 or —(CH2)ₚ-NR14-(C═O)—O—R15 where p represents an integer between 1 and 3 inclusive; or
a group having the formula

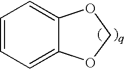

where q represents 1 or 2;
   or alternatively R1 and R2 together with the nitrogen atom to which they are attached form a heterocycloalkyl;
   R6 and R7 represent independently a hydrogen atom or an alkyl group;
   R14 and R15 represent independently a hydrogen atom or an alkyl group;
   or a pharmaceutically acceptable salt thereof.

When no further details are given, alkyl is understood to mean a linear or branched alkyl group containing 1 to 6 carbon atoms, such as methyl, ethyl, propyl, isopropyl, butyl, isobutyl, tert-butyl, pentyl or hexyl and preferably 1 to 4 carbon atoms.

Alkylamino or dialkylamino is understood in the present invention to mean an amino group substituted by one or two alkyl groups as hereinbefore defined, such as methylamino, dimethylamino, methylethylamino, ethylamino or diethylamino.

Aminoalkyl, alkylaminoalkyl or dialkylaminoalkyl is understood to mean an alkyl group as hereinbefore defined, substituted by an amino group or by an alkylamino or dialkylamino group as hereinbefore defined, such as dimethylaminoethyl or diethylaminoethyl.

Alkoxy is understood in the present invention to mean an —O-alkyl group where the alkyl moiety is as hereinbefore defined, such as the methoxy or ethoxy group.

Haloalkyl (or halogenoalkyl) is understood to mean an alkyl group as hereinbefore defined, substituted by one or more identical or different halogen atoms, such as trifluoromethyl or pentafluoroethyl.

Haloalkoxy (or haloalkyloxy or halogenoalkoxy) is understood to mean an —O-(haloalkyl) group where the haloalkyl group is as hereinbefore defined, such as the trifluoromethoxy group.

When no further details are given, cycloalkyl (or non-aromatic carbocycle) is understood to mean a saturated 3- to 7-membered cyclic carbon group, such as cyclopropyl, cyclobutyl, cyclopentyl, cyclohexyl or cycloheptyl and preferably cyclopentyl, cyclohexyl and cycloheptyl.

Heterocycloalkyl (or heterocyl) is understood in the present invention to mean a 3- to 6-membered ring including one or more identical or different heteroatoms selected from O, N and S, such as an azeridinyl, azetidinyl, pyrrolidinyl, piperidinyl, morpholinyl or tetrahydrofuran group.

Aryl (or aromatic carbocycle) is understood to mean an unsaturated carbocyclic system including at least one aromatic ring and preferably one moiety selected from phenyl, naphthyl and fluorenyl.

Arylalkyl is understood to mean an alkyl group as hereinbefore defined, substituted by an aryl group as hereinbefore defined, such as the benzyl group or the phenethyl group.

Heteroaryl is understood in the present invention to mean an unsaturated aromatic ring containing one or more identical or different heteroatoms selected from N, O and S such as pyridinyl, pyrimidinyl, furyl, thienyl oxazolyl, isoxazolyl, thiazolyl, pyrrolyl, pyrazolyl, imidazolyl, triazolyl, tetrazolyl and particularly tetrazolyl and pyridinyl.

Salt of a compound is understood to mean acid addition salts thereof with an organic or inorganic acid or, where appropriate, base addition salts, and in particular pharmaceutically acceptable salts of said compound.

Pharmaceutically acceptable salt is understood in particular to mean acid addition salts with inorganic acids such as hydrochloride, hydrobromide, hydroiodide, sulfate, phosphate, diphosphate and nitrate or with organic acids such as acetate, maleate, fumarate, tartrate, succinate, citrate, lactate, methanesulfonate, p-toluenesulfonate, pamoate and stearate. Salts formed from bases such as sodium hydroxide or potassium hydroxide also fall under the scope of the present invention, when they are usable. For other examples of pharmaceutically acceptable salts, refer to “Salt selection for basic drugs”, Int. J. Pharm. (1986), 33, 201-217.

The present invention relates preferably to a compound (I) as hereinabove defined where R4a, R5a, R4b and R5b are such that the —NR4aR5a and —NR4bR5b groups are identical.

Also preferably, the present invention relates to a compound (I) as hereinabove defined where R4a, R5a, R4b and R5b are such that the —NR4aR5a and —NR4bR5b groups are different.

Preferably, the present invention relates to a compound (I) as hereinabove defined and where:

Y represents independently an NR1R2 or OR13 group;
W represents independently —NR6- or —CR6R7-;
R3 represents a hydrogen atom;
n and m are integers from 0 to 2 inclusive;
R4a and R5a represent an alkyl group or alternatively together with the nitrogen atom to which they are attached form a heterocycloalkyl;
R4b and R5b represent an alkyl group or alternatively together with the nitrogen atom to which they are attached form a heterocycloalkyl;
R1 and R2 represent independently a hydrogen atom, or a group selected from:
alkyl;
arylalkyl optionally substituted by one or more identical or different halo groups;
heteroaryl;
aryl optionally substituted by one or more identical or different groups selected from: halo, hydroxy, cyano, nitro, alkyl, alkoxy, haloalkyl, aminoalkyl, alkylaminoalkyl, dialkylaminoalkyl, cycloalkyl, aryl, heteroaryl, —NH—(C═O)—R14, —(C═O)—R14 or —(CH2)ₚ-NR14-(C═O)—O—R15 where p represents an integer between 1 and 2 inclusive; or
a group having the formula

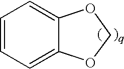

where q represents 1 or 2;
   or alternatively R1 and R2 together with the nitrogen atom to which they are attached form a heterocycloalkyl;
   R6 and R7 represent independently a hydrogen atom or an alkyl group;
   R13 represents a hydrogen atom or an alkyl group;
   R14 and R15 represent independently a hydrogen atom or an alkyl group.

More preferably, the invention relates to a compound (I) as hereinabove defined where:

Y represents an NR1R2 group;
W represents —CR6R7-;
R1 and R2 represent independently a hydrogen atom, or a group selected from:
alkyl,
arylalkyl optionally substituted by a halogen atom;
aryl optionally substituted by one or more identical or different groups selected from: halo, cyano, nitro, alkyl, alkoxy, haloalkyl, aryl, —(C═O)—R14 or —(CH2)ₚ-NR14-(C═O)—O—R15 where p represents an integer between 1 and 2 inclusive; or
a group having the formula

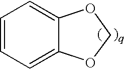

where q represents 1;
   R6 and R7 represent a hydrogen atom;
   and even more preferably a compound (I) where:
   R4a and R5a represent an alkyl group or alternatively together with the nitrogen atom to which they are attached form a heterocycloalkyl selected from pyrrolidine and piperidine;
   R4b and R5b represent an alkyl group or alternatively together with the nitrogen atom to which they are attached form a heterocycloalkyl selected from pyrrolidine and piperidine;
   R1 and R2 represent independently a hydrogen atom, or a group selected from alkyl, benzyl optionally substituted by a halogen atom, benzodioxole, or phenyl optionally substituted by one or more identical or different groups selected from halo, cyano, nitro, alkyl, alkoxy, trifluoromethyl, phenyl, —(C═O)—R14 or —(CH2)ₚ-NR14-(C═O)—O—R15 where p represents an integer between 1 and 2 inclusive;

Very preferably, the invention relates to a compound (I) as hereinabove defined where Y represents NR1R2, W represents —CR6R7-, R1 represents a hydrogen atom and R2 an aryl group optionally substituted by one or more identical or different groups selected from: halo, cyano, nitro, alkyl, alkoxy, haloalkyl, phenyl; and even more preferably, R2 represents an aryl group optionally substituted by one or more identical or different halo groups.

Also preferably, in the compounds (I) according to the invention, m and n never represent 0 at the same time.

Very preferably, the invention relates to a compound (I) as hereinabove defined where W represents —CR6R7-, R6 and R7 represent respectively a hydrogen atom, n represents an integer selected from 1 and 2, and m represents an integer selected from 0, 1 and 2.

In the compound (I) as hereinabove defined, in a preferred embodiment the term alkyl in alkyl, alkoxy, haloalkyl, haloalkoxy, aminoalkyl, alkylaminoalkyl, dialkylaminoalkyl and aralkyl groups represents a group selected from methyl, ethyl, propyl, isopropyl, butyl, isobutyl, and tert-butyl.

In the compound (I) as hereinabove defined, according to another preferred embodiment, the term aryl in aryl and aralkyl groups represents the phenyl group.

In the compound (I) as hereinabove defined, according to another preferred embodiment, the term heterocycloalkyl represents a group selected from pyrrolidino, piperidino, morpholino, and azetidino; and very preferably a pyrrolidine group.

In the compound (I) as hereinabove defined, according to another preferred embodiment, the term cycloalkyl represents a group selected from cyclopropyl, cyclobutyl, cyclopentyl, cyclohexyl and cycloheptyl.

In the compound (I) as hereinabove defined, according to another preferred embodiment, the term heteroaryl represents the pyridinyl, pyrrolyl, imidazolyl, triazolyl, tetrazolyl, thiazolyl, oxazolyl, thienyl, furyl or group; and very preferably a tetrazolyl or pyridinyl group.

The present invention also relates to compounds having the general formula (I′) below

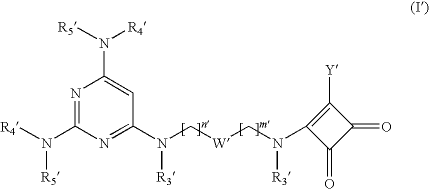

in a racemic form, an enantiomeric form or any combination thereof, where:
W′ represents independently NR₆′ or CR₆′R₇′ it being understood that R₆′ and R₇′ represent independently a hydrogen atom or a linear or C₁-C₆ branched alkyl group;
R₃′ represents a hydrogen atom or a linear or C₁-C₆ branched alkyl group;
or R₄′ and R₅′ together form a heterocycle including the nitrogen atom;
or R₄′ and R₅′ represent independently a hydrogen atom or a linear or C₁-C₆ branched alkyl group;
n′ or m′ is an integer between 0 and 4 inclusive;
Y′ represents either a NR₁′R₂′ group or alternatively an OR₁′ group;
R₁′ and R₂′ represent independently either a hydrogen atom, a linear or C₁-C₆ branched alkyl group, a benzyl group optionally substituted by a halogen atom, or a benzodioxole group;
or alternatively R₁′ or R₂′ represents independently a

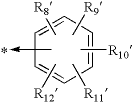

group where R₈′, R₉′, R₁₀′, R₁₁′ or R₁₂′ represent independently a hydrogen atom, a halogen atom, an OH, CN, NO₂, OCF₃, CF₃ group, an aminoalkyl group, an alkoxy group, a linear or C₁-C₆ branched alkyl group, a phenyl group, a tetrazole group, an NH—(C═O)—R₃′ group, a (—C═O)—R₃′ group, a —(CH2)ₚ-NR₃′—(C═O)—O—R₃′ group, a —(CH2)ₚ-NR₃′—R₃′ group, or a non-aromatic carbocycle group;
where p′ is an integer between 1 and 3 inclusive;
or alternatively
R₁′ and R₂′ together can form a heterocycle including the nitrogen atom;
or a pharmaceutically acceptable salt thereof.

Preferably, the compound according to the invention has R₄′ and R₅′ groups which together form a heterocycle including the nitrogen atom, and more particularly form a pyrrolidine group.

Preferably, the compound of the invention has a R₃′ group that represents a hydrogen atom.

Preferably, the compound of the invention is such that n′ or m′ is an integer equal to 1 or 2.

Preferably, the compound of the invention has a Y′ group that represents a NR₁′R₂′ group with R₂′ representing a hydrogen atom.

Preferably, when R₁′ and R₂′ together form a heterocycle including the nitrogen atom, the heterocycle is then preferably a morpholine or piperidine group.

Preferably, the compound according to the invention has a W′ group that represents a CR₆′R₇′ group with R₆′ and R₇′ each representing a hydrogen atom.

Preferably, the compound according to the invention has a Y′ group that represents an NR₁′R₂′ group with R₁′ representing a hydrogen atom and R₂′ representing a

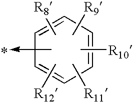

group where R₈′, R₉′, R₁₀′, R₁₁′ or R₁₂′ is defined above.

According to one embodiment, the invention relates to a compound having the general formula (I′) where W′ represents CR₆′R₇′; R₆′ and R₇′ represent a hydrogen atom; n′ or m′ is an integer between 0 and 2 inclusive, or a pharmaceutically acceptable salt thereof.

According to this embodiment, preferably, R₄′ and R₅′ together form a heterocycle including the nitrogen atom, and more particularly a pyrrolidine group.

According to this embodiment, preferably, R₃′ represents a hydrogen atom.

According to this embodiment, preferably, n′ is an integer equal to 1 and m′ represents 0.

According to this embodiment, preferably, Y′ represents an NR₁′R₂′ group with R₂′ representing a hydrogen atom and more particularly when R₁′ represents a

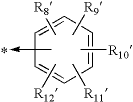

group.

According to this embodiment, preferably, R₈′, R₉′, R₁₀′, R₁₁′ and R₁₂′ represent independently and preferably either one or several hydrogen atoms, or one or several halogen atoms, or one or several CF₃ or NO₂ groups, or alternatively a phenyl group.

The terminology used for the nomenclature of the compounds hereinabove, for the examples and more generally throughout the text is the English IUPAC terminology.

In addition, some of the compounds having the general formula (I) or (I′) can be in an enantiomeric form. The present invention includes both enantiomeric forms and any combinations thereof, including “R,S” racemic mixtures. For the sake of simplicity, when no specific configuration is indicated in the structural formulae, it should be taken to mean that both enantiomeric forms and mixtures thereof are represented.

The invention preferably relates to a compound as hereinabove defined selected from the following compounds:
3-({2-[(2,6-dipyrrolidin-1-ylpyrimidin-4-yl)amino]ethyl}amino)-4-methoxycyclobut-3-ene-1,2-dione;
3-({2-[(2,6-dipyrrolidin-1-ylpyrimidin-4-yl)amino]ethyl}amino)-4-piperidin-1-ylcyclobut-3-ene-1,2-dione;
3-(butylamino)-4-({2-[(2,6-dipyrrolidin-1-ylpyrimidin-4-yl)amino]ethyl}amino) cyclobut-3-ene-1,2-dione;
3-({2-[(2,6-dipyrrolidin-1-ylpyrimidin-4-yl)amino]ethyl}amino)-4-morpholin-4-ylcyclobut-3-ene-1,2-dione;
3-[(4-chlorobenzyl)amino]-4-({2-[(2,6-dipyrrolidin-1-ylpyrimidin-4-yl)amino]ethyl}amino)cyclobut-3-ene-1,2-dione;
3-({2-[{2-[(2,6-dipyrrolidin-1-ylpyrimidin-4-yl)amino]ethyl}(methyl)amino]ethyl}amino)-4-methoxycyclobut-3-ene-1,2-dione;
3-({2-[{2-[(2,6-dipyrrolidin-1-ylpyrimidin-4-yl)amino]ethyl}(methyl)amino]ethyl}amino)-4-hydroxycyclobut-3-ene-1,2-dione;
3-[(4-chlorophenyl)amino]-4-({2-[{2-[(2,6-dipyrrolidin-1-ylpyrimidin-4-yl)amino]ethyl}(methyl)amino]ethyl}amino)cyclobut-3-ene-1,2-dione;
3-[(4-chlorophenyl)amino]-4-({2-[(2,6-dipyrrolidin-1-ylpyrimidin-4-yl)amino]ethyl}amino)cyclobut-3-ene-1,2-dione;
3-{[4-chloro-3-(trifluoromethyl)phenyl]amino}-4-({2-[(2,6-dipyrrolidin-1-ylpyrimidin-4-yl)amino]ethyl}amino)cyclobut-3-ene-1,2-dione;
3-(biphenyl-4-ylamino)-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-{[3-(1H-tetrazol-5-yl)phenyl]amino}cyclobut-3-ene-1,2-dione;
N-(4-{[2-({2-[(2,6-dipyrrolidin-1-ylpyrimidin-4-yl)amino]ethyl}amino)-3,4-dioxocyclobut-1-en-1-yl]amino}phenyl)acetamide;
3-({2-[(2,6-dipyrrolidin-1-ylpyrimidin-4-yl)amino]ethyl}amino)-4-[(3,4,5-trimethoxyphenyl)amino]cyclobut-3-ene-1,2-dione;
3-[(3,5-difluorophenyl)amino]-4-({2-[(2,6-dipyrrolidin-1-ylpyrimidin-4-yl)amino]ethyl}amino)cyclobut-3-ene-1,2-dione;
3-[(2-chlorophenyl)amino]-4-({2-[(2,6-dipyrrolidin-1-ylpyrimidin-4-yl)amino]ethyl}amino)cyclobut-3-ene-1,2-dione;
3-[(3-chlorophenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-methoxyphenyl)amino]cyclobut-3-ene-1,2-dione;
3-[(4-acetylphenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(3-methoxyphenyl)amino]cyclobut-3-ene-1,2-dione;
tert-butyl (4-{[2-({2-[(2,6-dipyrrolidin-1-ylpyrimidin-4-yl)amino]ethyl}amino)-3,4-dioxocyclobut-1-en-1-yl]amino}benzyl)methylcarbamate;
3-({2-[(2,6-dipyrrolidin-1-ylpyrimidin-4-yl)amino]ethyl}amino)-4-({4-[(methylamino)methyl]phenyl}amino)cyclobut-3-ene-1,2-dione;
3-[(4-cyclohexylphenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-propylphenyl)amino]cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)carbamate;
3-(1,3-benzodioxol-5-ylamino)-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-hydroxyphenyl)amino]cyclobut-3-ene-1,2-dione;
3-{[4-(aminomethyl)phenyl]amino}-4-({2-[(2,6-dipyrrolidin-1-ylpyrimidin-4-yl)amino]ethyl}amino)cyclobut-3-ene-1,2-dione;
tert-butyl[2-(4-{[2-({2-[(2,6-dipyrrolidin-1-ylpyrimidin-4-yl)amino]ethyl}amino)-3,4-dioxocyclobut-1-en-1-yl]amino}phenyl)ethyl]carbamate;
3-{[4-(2-aminoethyl)phenyl]amino}-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-fluorophenyl)amino]cyclobut-3-ene-1,2-dione;
3-({2-[(2,6-dipyrrolidin-1-ylpyrimidin-4-yl)amino]ethyl}amino)-4-{[4-(trifluoromethyl)phenyl]amino}cyclobut-3-ene-1,2-dione;
3-({2-[(2,6-dipyrrolidin-1-ylpyrimidin-4-yl)amino]ethyl}amino)-4-[(4-cyanophenyl)amino]cyclobut-3-ene-1,2-dione;
3-({2-[(2,6-dipyrrolidin-1-ylpyrimidin-4-yl)amino]ethyl}amino)-4-[(4-nitrophenyl)amino]cyclobut-3-ene-1,2-dione;
3-anilino-4-({2-[(2,6-dipyrrolidin-1-ylpyrimidin-4-yl)amino]ethyl}amino)cyclobut-3-ene-1,2-dione;
3-[(4-chloro-3-fluorophenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-(pyridin-3-ylamino)cyclobut-3-ene-1,2-dione;
3-[(4-chlorophenyl)amino]-4-[(2-{[2-(diethylamino)-6-pyrrolidin-1-ylpyrimidin-4-yl]amino}ethyl)amino]cyclobut-3-ene-1,2-dione;
   or a salt thereof.

The invention very preferably relates to a compound as hereinabove defined selected from the following compounds:
3-[(4-chlorobenzyl)amino]-4-({2-[(2,6-dipyrrolidin-1-ylpyrimidin-4-yl)amino]ethyl}amino)cyclobut-3-ene-1,2-dione;
3-[(4-chlorophenyl)amino]-4-({2-[(2,6-dipyrrolidin-1-ylpyrimidin-4-yl)amino]ethyl}amino)cyclobut-3-ene-1,2-dione;
3-(biphenyl-4-ylamino)-4-({2-[(2,6-dipyrrolidin-1-ylpyrimidin-4-yl)amino]ethyl}amino)cyclobut-3-ene-1,2-dione;
3-[(3,5-difluorophenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-methoxyphenyl)amino]cyclobut-3-ene-1,2-dione;
3-[(4-acetylphenyl)amino]-4-({2-[(2,6-dipyrrolidin-1-ylpyrimidin-4-yl)amino]ethyl}amino)cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)methylcarbamate;
3-({2-[(2,6-dipyrrolidin-1-ylpyrimidin-4-yl)amino]ethyl}amino)-4-[(4-propylphenyl)amino]cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)carbamate;
3-(1,3-benzodioxol-5-ylamino)-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-fluorophenyl)amino]cyclobut-3-ene-1,2-dione;
3-({2-[(2,6-dipyrrolidin-1-ylpyrimidin-4-yl)amino]ethyl}amino)-4-{[4-(trifluoromethyl)phenyl]amino}cyclobut-3-ene-1,2-dione;
3-({2-[(2,6-dipyrrolidin-1-ylpyrimidin-4-yl)amino]ethyl}amino)-4-[(4-cyanophenyl)amino]cyclobut-3-ene-1,2-dione;
3-({2-[(2,6-dipyrrolidin-1-ylpyrimidin-4-yl)amino]ethyl}amino)-4-[(4-nitrophenyl)amino]cyclobut-3-ene-1,2-dione;
3-[(4-chloro-3-fluorophenyl)amino]-4-({2-[(2,6-dipyrrolidin-1-ylpyrimidin-4-yl)amino]ethyl}amino)cyclobut-3-ene-1,2-dione;
3-[(4-chlorophenyl)amino]-4-[(2-{[2-(diethylamino)-6-pyrrolidin-1-ylpyrimidin-4-yl]amino}ethyl)amino]cyclobut-3-ene-1,2-dione;
   or a salt thereof,
   and more preferably still the compound 3-(4-chlorophenyl)amino-4-({2-(2,6-dipyrrolidin-1-ylpyrimidin-4-yl)aminoethyl}amino)cyclobut-3-ene-1,2-dione.

Using the definitions given hereinbefore for the variable groups R3, R4a, R5a, R4b, R5b, Y, m, n, and W, the compounds of the invention can be prepared according to the various procedures described below.

1) Preparation of the Intermediate Having the Formula (IV):

The intermediate having the general formula (IV) may be synthesised in two ways depending on the nature of the groups R4a, R5a, R4b and R5b:
a) When the groups —NR4aR5a and —NR4bR5b, are different, the amines HNR4aR5a and HNR4bR5b react successively to obtain the compound having the general formula IV according to the following reaction scheme A:

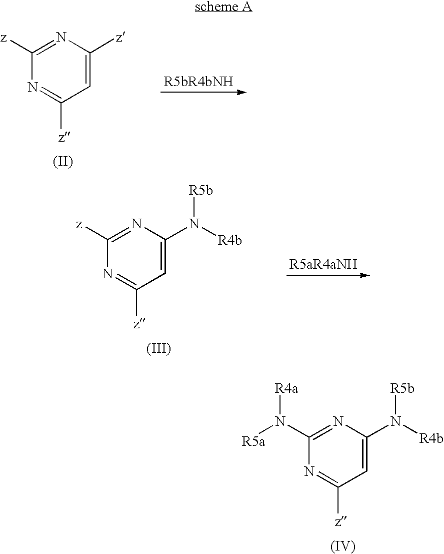

z, z′ and z″ represent independently a halogen atom and preferably a chlorine atom.

The diaminopyrimidine derivatives having the general (formula IV) can be prepared in two synthetic steps by reacting for example compound (II) with the amine of general formula R5bR4bNH where R4b and R5b are as hereinbefore defined, at a temperature between −5° C. and 5° C. (preferably 0° C.), in an inert solvent such as tetrahydrofuran. The resulting compound (III) reacts with the amine of general formula R5aR4aNH where R4a and R5a are as hereinbefore defined, at a temperature between 50° C. and 70° C. (preferably 65° C.) in an inert polar solvent such as tetrahydrofuran.
b) When the group —NR4aR5a is identical to the group —NR4bR5b, the same amine HNR4aR5a or HNR4bR5b can be used to obtain the compound IV according to the following reaction scheme B:

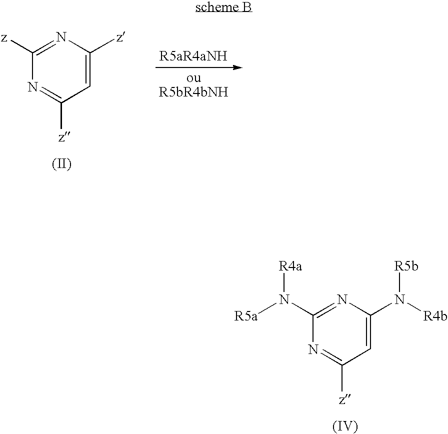

z, z′ and z″ represent independently a halogen atom and preferably a chlorine atom.

The diaminopyrimidine derivatives having the general formula (IV) can be prepared according to the method described by Bundy et al. in Journal of Medicinal Chemistry, 1995, 35, 4161-4163 by reacting for example compound (II) with the amine of general formula R5aR4aNH or R5bR4bNH where R4a, R5a, R4b and R5b are as hereinbefore defined, at a temperature between −5° C. and 5° C. (preferably 0° C.) in an inert solvent such as tetrahydrofuran.

In the particular case where R4a, R5a, R4b and R5b all represent a methyl group and in the particular case where the groups —NR4aR5a and NR4bR5b represent an ethylamino group, the conditions for preparing derivatives having the formula (IV) are as previously described by Atri et al. in Journal of Medicinal Chemistry, 1984, 27, 1621-1629. The reaction occurs at a temperature between 30° C. and 50° C. (preferably 40° C.) in an inert polar solvent such as ethanol.

2) Preparation of the Intermediate Having the Formula (VI):

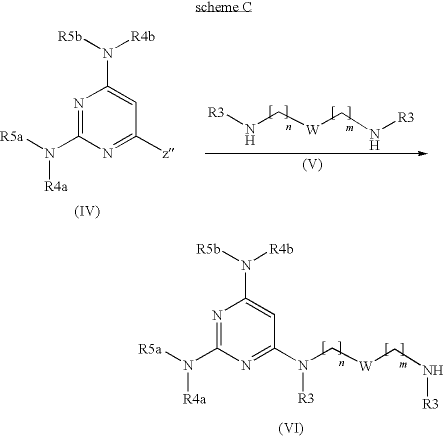

As described in scheme C hereinabove, the compounds of general formula (VI) where R3, R4a, R5a, R4b, R5b, W, n and m are as hereinbefore defined, can be obtained, for example, by heating or microwave heating the compound of formula (IV) to a temperature between 150° C. and 250° C. (preferably 190° C.), with a large excess of the diamine compound (V).

3) Preparation of the Compounds Having the Formula (I):

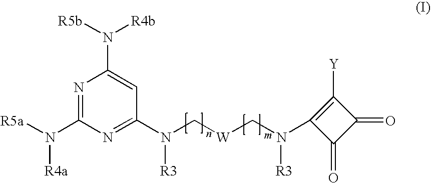

Depending on the nature of group Y, there can be several embodiments of the preparation of the compounds having the general formula (I) to produce respectively compounds having the formula (I) where Y=OR13 (Ia), Y=OH (Ib) and Y=NR1R2 (Ic) as described below.

3a) Preparation of the Compounds Having Formula (Ia) and (Ib):

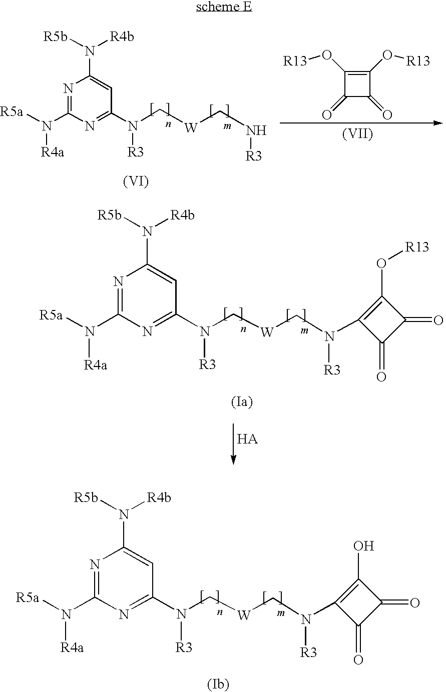

The derivatives having the general formula (Ia) where R3, R4a, R5a, R4b, R5b, R13, W, n, and m are as hereinbefore defined, can be prepared according to the method described in scheme E by reacting the intermediate compound (VI) with the cyclobut-3-ene-1,2-dione derivative of general formula (VII) at a temperature between 10° C. and 30° C. (preferably 20° C.) in an inert polar solvent such as methanol. The compound (Ib) may be obtained by an acid hydrolysis reaction of compound (Ia) using for example an inorganic acid of formula HA such as dilute hydrochloric acid at a temperature between 50° C. and 70° C. (preferably 65° C.) in an inert polar solvent such as methanol.

3b) Preparation of the Compound Having the Formula (Ic):

The compounds having the general formula (Ic) can be prepared by two synthetic routes:

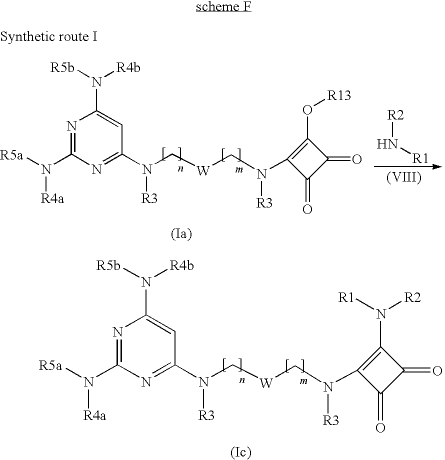

The derivatives having the general formula (Ic) where R1, R2, R4a, R5a, R4b, R5b, W, n and m are as hereinbefore defined, can be prepared by the method described in scheme F by reacting the compound (Ia) as hereinabove defined with the amine of general formula (VIII) at a temperature between 10° C. and 30° C. (preferably 20° C.) in an inert polar solvent such as ethanol.

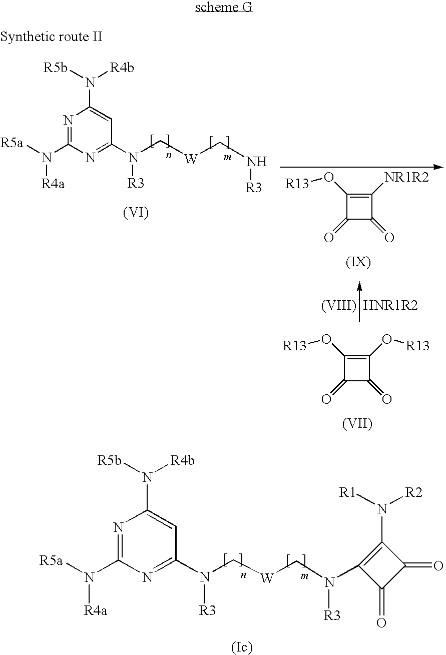

As described in scheme G above, the compounds having the general formula (Ic) where R1, R2, R3, R4a, R5a, R4b, R5b, W, n and m are as hereinbefore defined, can be obtained by heating the aminocyclobut-3-ene-1,2-dione derivative of general formula (IX) with the intermediate derivative of general formula (VI) at a temperature between 50° C. and 70° C. (preferably 60° C.) in a polar solvent such as ethanol.

The 4-membered cyclic derivative having the formula (IX) may be obtained by reacting the amine derivative of general formula (VIII) and the cyclobut-3-ene-1,2-dione derivative of formula (VII) by heating to the reflux temperature of a polar solvent such as ethanol, at a temperature between 70° C. and 90° C. (preferably 80° C.).

This second synthetic route is used preferentially in cases where one of the groups R1 or R2 is an aromatic group.

When the aromatic group has an aminoalkyl or alkylaminoalkyl substituent, the compound is obtained from the corresponding compound where the amine group is protected by a tert-butylcarbamate type group using methods well know to the skilled person.

The present invention also relates to an industrial compound that is a synthetic intermediate selected from the following compounds:
3-[(4-chlorophenyl)amino]-4-methoxycyclobut-3-ene-1,2-dione;
3-([4-chloro-3-(trifluoromethyl)phenyl]amino)-4-methoxycyclobut-3-ene-1,2-dione;
3-(biphenyl-4-ylamino)-4-methoxycyclobut-3-ene-1,2-dione;
3-methoxy-4-{[4-(2H-tetrazol-5-yl)phenyl]amino}cyclobut-3-ene-1,2-dione;
N-{4-[(2-methoxy-3,4-dioxocyclobut-1-en-1-yl)amino]phenyl}acetamide;
3-methoxy-4-[(3,4,5-trimethoxyphenyl)amino]cyclobut-3-ene-1,2-dione;
3-[(3,5-difluorophenyl)amino]-4-methoxycyclobut-3-ene-1,2-dione;
3-[(2-chlorophenyl)amino]-4-methoxycyclobut-3-ene-1,2-dione;
3-[(3-chlorophenyl)amino]-4-methoxycyclobut-3-ene-1,2-dione;
3-methoxy-4-[(4-methoxyphenyl)amino]cyclobut-3-ene-1,2-dione;
3-[(4-acetylphenyl)amino]-4-methoxycyclobut-3-ene-1,2-dione;
tert-butyl{4-[(2-methoxy-3,4-dioxocyclobut-1-en-1-yl)amino]benzyl}methylcarbamate;
3-[(4-cyclohexylphenyl)amino]-4-methoxycyclobut-3-ene-1,2-dione;
3-methoxy-4-[(4-propylphenyl)amino]cyclobut-3-ene-1,2-dione;
tert-butyl {4-[(2-methoxy-3,4-dioxocyclobut-1-en-1-yl)amino]benzyl}carbamate;
3-(1,3-benzodioxol-5-ylamino)-4-methoxycyclobut-3-ene-1,2-dione;
3-[(4-hydroxyphenyl)amino]-4-methoxycyclobut-3-ene-1,2-dione;
tert-butyl(2-{4-[(2-methoxy-3,4-dioxocyclobut-1-en-1-yl)amino]phenyl}ethyl)carbamate;
3-methoxy-4-{[4-(trifluoromethyl)phenyl]amino}cyclobut-3-ene-1,2-dione;
4-[(2-methoxy-3,4-dioxocyclobut-1-en-1-yl)amino]benzonitrile;
3-methoxy-4-[(4-nitrophenyl)amino]cyclobut-3-ene-1,2-dione;
3-[(4-chloro-3-fluorophenyl)amino]-4-methoxycyclobut-3-ene-1,2-dione;
3-methoxy-4-(pyridin-3-ylamino)cyclobut-3-ene-1,2-dione.

The invention also relates to a process for preparing a compound having the general formula (I) as hereinabove defined from compounds having the general formula (VI)

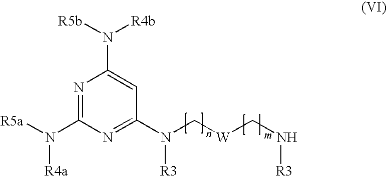

where R3, R4a, R5a, R4b, R5b, W, m and n are as hereinabove defined and according to which:
a) either the compound of general formula (VI) as hereinabove defined is reacted with a compound of general formula (VII)

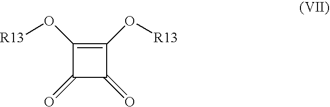

where R13 is as hereinbefore defined to obtain the compound having the general formula (I) where Y represents OR13;
and the compound of general formula (I) where Y represents OR13 and R13 represents an alkyl group can be reacted:
either with an inorganic acid to form the compound of general formula (I) where Y represents OR13 and R13 represents a hydrogen atom;
or with an amine of general formula HNR1R2 where R1 and R2 are as hereinbefore defined to form the compound of general formula (I) where Y represents NR1R2;
b) or the compound of general formula (VI) as hereinabove defined can be reacted with a compound of general formula (IX)

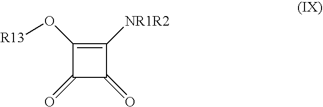

where R1, R2 and R13 are as hereinbefore defined to obtain the compound of general formula (I) where Y represents NR1R2.

The present invention also relates to a compound having the general formula (I) or (I′) as hereinabove defined or a salt thereof, for use as a therapeutically active substance.

The present invention also relates to a pharmaceutical composition containing, as an active substance, a compound having the general formula (I) or (I′) as hereinabove defined, or a pharmaceutically acceptable salt of such a compound, with at least one pharmaceutically acceptable excipient.

The present invention also relates to a compound having the general formula (I) or (I′) as hereinabove defined, or a pharmaceutically acceptable salt of such a compound, as a drug.

The present invention also relates to the use of at least one compound having the general formula (I) or (I′) as hereinabove defined or a pharmaceutically acceptable salt of such a compound, to prepare a drug intended for the treatment or prevention of a disease or disorder selected from the following diseases or disorders: cancer, cancerous proliferative diseases, noncancerous proliferative diseases, neurodegenerative diseases, parasitic diseases, viral infections, spontaneous alopecia, alopecia induced by exogenous products, radiation-induced alopecia, autoimmune diseases, graft rejection, inflammatory diseases or allergies.

More particularly, the present invention relates to the use of at least one compound having the general formula (I) or (I′) as hereinabove defined or a pharmaceutically acceptable salt of such a compound, to prepare a drug intended for the treatment or prevention of cancer.

Even more particularly, the present invention relates to the use of at least one compound having the general formula (I) or (I′) as hereinabove defined or a pharmaceutically acceptable salt of such a compound, to prepare a drug intended for the treatment or prevention of cancer, said cancer being selected from cancer of the colon, rectum, stomach, lung, pancreas, kidney, testicle, breast, uterus, ovary, prostate, skin, bone, spinal cord, neck, tongue or head, as well as sarcomas, carcinomas, fibroadenomas, neuroblastomas, leukaemias and melanomas.

The present invention also relates to the use of at least one compound having the general formula (I) or (I′) as hereinabove defined or a pharmaceutically acceptable salt of such a compound, for the treatment or prevention of a disease or disorder selected from the following diseases or disorders: cancers, cancerous proliferative diseases, noncancerous proliferative diseases, neurodegenerative diseases, parasitic diseases, viral infections, spontaneous alopecia, alopecia induced by exogenous products, radiation-induced alopecia, autoimmune diseases, graft rejection, inflammatory diseases or allergies.

More particularly, the present invention relates to the use of at least one compound having the general formula (I) or (I′) as hereinabove defined or a pharmaceutically acceptable salt of such a compound, for the treatment or prevention of cancer.

Even more particularly, the present invention relates to the use of at least one compound having the general formula (I) or (I′) as hereinabove defined or a pharmaceutically acceptable salt of such a compound, for the treatment or prevention of cancer, said cancer being selected from cancer of the colon, rectum, stomach, lung, pancreas, kidney, testicle, breast, uterus, ovary, prostate, skin, bone, spinal cord, neck, tongue or head, as well as sarcomas, carcinomas, fibroadenomas, neuroblastomas, leukaemias and melanomas.

The compound having the general formula (I) or (I′) or a salt thereof used according to the invention may be in the form of a solid, for example powders, granules, tablets, capsules, liposomes or suppositories. Suitable solid bases may be, for example, calcium phosphate, magnesium stearate, talc, sugars, lactose, dextrin, starch, gelatin, cellulose, methylcellulose, sodium carboxymethylcellulose, polyvinylpyrrolidine and wax.

The compound having the general formula (I) or (I′) or a salt thereof used according to the invention or the combination according to the invention may exist in liquid form, for example solutions, emulsions, suspensions or syrups. Suitable liquid bases may be, for example, water, organic solvents such as glycerol or glycols, or blends thereof, in varying proportions, in water.

A compound having the general formula (I) or (I′) or a salt thereof used according to the invention or the combination according to the invention can be administered topically, orally, parenterally, by intramuscular injection, by subcutaneous injection etc.

The anticipated dose of a product according to the present invention for the treatment of the diseases or disorders mentioned hereinabove varies depending on the method of administration, the age and the body weight of the subject to be treated as well as the subject's condition, and in the end will be decided by the treating doctor or veterinarian. Such a quantity determined by the treating doctor or veterinarian is referred to herein as the “therapeutically effective quantity”.

As an indication only, the envisaged dose of a drug according to the invention is between 0.1 mg and 10 g depending on the type of active compound used.

### EXPERIMENTAL PART

The NMR analyses of examples 1 through 38 were performed on a 400 MHz Bruker-Avance II spectrometer.

The compounds are characterised by their molecular (MH+) peak determined by mass spectrometry (MS), a single quadrupole mass spectrometer (Micromass, Platform model) equipped with an electrospray source is used with a resolution of 0.8 Da (50% valley definition). For examples 1 through 38 below, the elution conditions corresponding to the indicated results are as follows: elution with an acetonitrile-water-trifluoroacetic acid mixture 50-950-0.2 (A) for 1 minute, switching from mixture (A) to an acetonitrile-water mixture 950-50 (B) by a linear gradient over a period of 7.5 minutes, followed by elution with pure mixture B for 2 minutes.

Using the definitions given hereinbefore for the variable groups R3, R4a, R5a, R4b, R5b, Y, m, n and W, the compounds of the invention can be prepared according to the various procedures described hereinabove.

The examples are presented to illustrate the above procedures and should under no circumstances be considered to limit the scope of the invention.

### Example 1

### 3-({2-[(2,6-dipyrrolidin-1-ylpyrimidin-4-yl)amino]ethyl}amino)-4-methoxycyclobut-3-ene-1,2-dione

### 1-1) 4-chloro-2,6-dipyrrolidin-1-ylpyrimidine

The compound 2,4,6-trichloropyrimidine (30 g, 164 mmol) is added to a solution containing pyrrolidine (44 ml, 524 mmol) in 60 ml of tetrahydrofuran at a temperature of 0° C. The reaction mixture is stirred for 2 hours at this temperature and then for 12 hours at 23° C. Then 15 ml of pyridine is added and stirring is maintained for one half-day. 60 ml of water are added, then the reaction mixture is extracted with 3×30 ml of dichloromethane. The resulting organic phase is poured into ice-cold water then neutralised with a saturated solution of sodium bicarbonate then with a saturated solution of sodium chloride. The organic phase is dried over sodium sulfate and the solvent is then eliminated using a rotary evaporator. The resulting oil is applied to a Biotage type chromatography column containing silica (eluent: ethyl acetate-heptane: 0-100 to 5-95) and a solid is obtained in the form of a white powder. The yield of the reaction is 66%.

¹H-NMR (δ ppm, DMSO): 1.84-1.87 (m, 8H); 3-3.39 (m, 8H); 5.74 (s, 1H)

Observed MH+=253.20; theoretical M=252.12

Melting point: 84-86° C.

### 1-2) N-(2,6-dipyrrolidin-1-ylpyrimidin-4-yl)ethane-1,2-diamine

In a sealed glass tube suitable for microwave heating, the compound 4-chloro-2,6-dipyrrolidin-1-ylpyrimidine as prepared in section 1-1) (0.4 g, 1.58 mmol) and ethylenediamine (1.5 ml, 20 mmol) are heated in a microwave oven (Biotage, Emrys Optimizer) at 190° C. for 3600 seconds. When the reaction is complete, 20 ml of water are added then the reaction mixture is extracted with ethyl acetate. It is washed with 3×20 ml of water then the organic phase is dried over sodium sulfate. It is evaporated to dryness and then about 10 ml of heptane is added to the resulting oil. After stirring, the resulting solid is filtered with a sintered-glass filter. A solid is obtained in the form of a white powder. The yield of the reaction is 70%.

¹H-NMR (δ ppm, CDCl₃): 1.87-1.94 (m, 8H); 2.80-2.90 (m, 2H); 3.40-3.60 (m, 12H); 4.60-4.65 (se, 1H); 4.80 (s, 1H)

Observed MH+=277.30; theoretical M=276.38

### 1-3) 3-({2-[(2,6-dipyrrolidin-1-ylpyrimidin-4-yl)amino]ethyl}amino)-4-methoxycyclobut-3-ene-1,2-dione

A mixture containing N-(2,6-dipyrrolidin-1-ylpyrimidin-4-yl)ethane-1,2-diamine as prepared in section 1-2) (0.46 g, 1.16 mmol) and 3,4-dimethoxycyclobut-3-ene-1,2-dione (0.5 g, 3.52 mmol) in 7 ml of methanol is stirred for 3 hours at 23° C. After eliminating the solvent using a rotary evaporator, the residual oil is purified by chromatography on a Biotage type silica column (eluent: dichloromethane-methanol-ammonia: 96-3-1) and a solid is obtained in the form of an orange powder. This powder is triturated in a minimum of ether then filtered with a sintered-glass filter. It is washed with ether. After drying, a solid is obtained in the form a pale brown powder. The yield of the reaction is 53%.

¹H-NMR (δ ppm, DMSO): 1.64-1.68 (m, 8H); 3.40-3.60 (m, 10H); 3.81-3.83 (m, 2H); 4.32-4.77 (m, 5H); 8.20 (m, 1H)

Observed MH+=387.35; theoretical M=386.21

Melting point: 116-118° C.

### Example 2

### 3-({2-[(2,6-dipyrrolidin-1-ylpyrimidin-4-yl)amino]ethyl}amino)-4-piperidin-1-ylcyclobut-3-ene-1,2-dione

A mixture containing 3-({2-[(2,6-dipyrrolidin-1-ylpyrimidin-4-yl)amino]ethyl}amino)-4-methoxycyclobut-3-ene-1,2-dione as prepared in example 1 (0.1 g, 0.26 mmol) and piperidine (0.022 g, 0.26 mmol) in 5 ml of ethanol is stirred for 3 hours at 23° C. The solid formed is filtered using a sintered-glass filter and washed with ether. After drying, a solid is obtained in the form of a beige powder. The yield of the reaction is 70%.

¹H-NMR (δ ppm, DMSO): 1.54-1.62 (m, 6H); 1.90-1.94 (m, 8H); 3.39-3.50 (m, 12H); 3.68-3.71 (m, 2H); 3.90-3.94 (m, 2H); 4.52 (se, 1H); 4.76 (s, 1H); 6.83 (se, 1H)

Observed MH+=440.39; theoretical M=439.27

Melting point: >250° C.

### Example 3

### 3-(butylamino)-4-({2-[(2,6-dipyrrolidin-1-ylpyrimidin-4-yl)amino]ethyl}amino)cyclobut-3-ene-1,2-dione

The compound of example 3 was synthesised according to a method analogous to the one described in example 2, using the compound from example 1, by reaction with butylamine.

¹H-NMR (δ ppm, CDCl₃): 0.86-0.89 (m, 3H); 1.20-1.27 (m, 4H); 1.93 (m, 8H); 3.40-3.54 (m, 12H); 3.81-3.83 (m, 2H); 4.30 (se, 1H); 4.78 (s, 1H); 5.50 (se, 1H); 7.00 (se, 1H)

Observed MH+=428.36; theoretical M=427.27

Melting point: 246-248° C.

### Example 4

### 3-({2-[(2,6-dipyrrolidin-1-ylpyrimidin-4-yl)amino]ethyl}amino)-4-morpholin-4-ylcyclobut-3-ene-1,2-dione

The compound of example 4 was synthesised according to a method analogous to the one described in example 2, using the compound from example 1, by reaction with morpholine.

¹H-NMR (δ ppm, CDCl₃): 1.91-1.94 (m, 8H); 3.39-3.93 (m, 20H); 4.50 (se, 1H); 4.77 (s, 1H); 7.30 (se, 1H)

Observed MH+=442.36; theoretical M=441.25

Melting point: 242-244° C.

### Example 5

### 3-[(4-chlorobenzyl)amino]-4-({2-[(2,6-dipyrrolidin-1-ylpyrimidin-4-yl)amino]ethyl}amino)cyclobut-3-ene-1,2-dione

The compound of example 5 was synthesised according to a method analogous to the one described in example 2, using the compound from example 1, by reaction with 4-chlorobenzylamine.

¹H-NMR (δ ppm, DMSO): 1.57 (m, 8H); 1.81-1.94 (m, 4H); 3.40-3.50 (m, 12H); 4.58 (se, 1H); 4.74 (s, 1H); 7.11 (m, 1H); 7.30 (se, 5H)

Observed MH+=496.26; theoretical M=495.21

Melting point: 246-248° C.

### Example 6

### 3-({2-[({2-[(2,6-dipyrrolidin-1-ylpyrimidin-4-yl)amino]ethyl}(methyl)amino]ethyl}amino)-4-methoxycyclobut-3-ene-1,2-dione

This compound is prepared according to a method analogous to example 1 described above. A solid is obtained in the form of a pale yellow powder. The yield of the reaction is 33%.

¹H-NMR (δ ppm, DMSO): 1.81-1.85 (m, 8H); 2.12-2.20 (m, 4H); 2.53-2.59 (m, 4H); 3.11 (m, 2H); 3.34-3.61 (m, 10H); 4.26-4.34 (m, 3H); 4.60 (s, 1H); 5.33 (se, 1H)

Observed MH+=444.37; theoretical M=443.26

Melting point: 132-134° C.

### Example 7

### 3-({2-[{2-[(2,6-dipyrrolidin-1-ylpyrimidin-4-yl)amino]ethyl}(methyl)amino]ethyl}amino)-4-hydroxycyclobut-3-ene-1,2-dione

Hydrochloric acid at a concentration of 1N (2.8 ml) is added at 23° C. to the compound 3-({2-[{2-[(2,6-dipyrrolidin-1-ylpyrimidin-4-yl)amino]ethyl}(methyl)amino]ethyl}amino)-4-methoxycyclobut-3-ene-1,2-dione as prepared in example 6 (0.206 g, 0.46 mmol) in 12 ml of methanol. The reaction medium is heated for 5 hours at 65° C. After eliminating the solvent from the reaction medium using a rotary evaporator, the residual oil is taken up in ether. It is evaporated again and this operation is repeated twice. The solid formed is filtered using a sintered-glass filter and washed with ether. After drying, a solid is obtained in the form of a dark green gum.

Observed MH+=430.33; theoretical M=429.25

### Example 8

### 3-[(4-chlorophenyl)amino]-4-({2-[{2-[(2,6-dipyrrolidin-1-ylpyrimidin-4-yl)amino]ethyl}(methyl)amino]ethyl}amino)cyclobut-3-ene-1,2-dione

### 8-1) N-{2-[(2,6-dipyrrolidin-1-ylpyrimidin-4-yl)amino]ethyl}-N-methylethane-1,2-diamine

In a sealed glass tube suitable for microwave heating, the compound 4-chloro-2,6-dipyrrolidin-1-ylpyrimidine as prepared in section (1-1) (0.8 g, 3.2 mmol) and N-methyl ethylenediamine (3.3 ml, 26 mmol) are heated in a microwave oven (Biotage, Emrys Optimizer) at 190° C. for 3600 seconds. When the reaction is complete, 20 ml of water are added and the reaction mixture is then extracted with ethyl acetate. It is washed with 3×20 ml of water then the organic phase is dried over sodium sulfate. It is evaporated to dryness and about 10 ml of heptane is then added to the resulting oil. After stirring, the resulting solid is filtered using a sintered-glass filter. A solid is obtained in the form of a white powder. The yield of the reaction is 69%.

¹H-NMR (δ ppm, DMSO): 1.61 (se, 2H); 1.87-1.94 (m, 8H); 2.23-2.25 (m, 3H); 2.43-2.46 (m, 2H); 2.58-2.61 (m, 2H); 2.76-2.79 (m, 2H); 3.28-3.30 (m, 2H); 3.42-3.54 (m, 8H); 4.75 (s, 1H); 4.80-4.85 (m, 1H)

Observed MH+=334.35; theoretical M=333.26

Melting point: 67-69° C.

### 8-2) 3-[(4-chlorophenyl)amino]-4-methoxycyclobut-3-ene-1,2-dione

A mixture containing 3,4-dimethoxycyclobut-3-ene-1,2-dione (0.78 g, 5.5 mmol) and 4-chloroaniline (0.23 g, 1.8 mmol) in 10 ml of methanol is stirred for 2 hours at 65° C. The resulting solid is filtered using a sintered-glass filter and washed with di-isopropyl ether. After drying, a solid is obtained in the form of a pale yellow powder. The yield of the reaction is 70%.

The resulting compound 8-2 can be used to produce the final compound 8-3 below by reacting compound 8-1) above with said compound 8-2.

¹H-NMR (δ ppm, DMSO): 4.37 (s, 3H); 7.37-7.42 (m, 4H); 10.81 (se, 1H)

Observed MH+=238.04; theoretical M=237.02

### 8-3) 3-[(4-chlorophenyl)amino]-4-({2-[{2-[(2,6-dipyrrolidin-1-ylpyrimidin-4-yl)amino]ethyl}(methyl)amino]ethyl}amino)cyclobut-3-ene-1,2-dione

A mixture containing N-{2-[(2,6-dipyrrolidin-1-ylpyrimidin-4-yl)amino]ethyl}-N-methylethane-1,2-diamine as prepared in section (8-1) (0.089 g, 0.32 mmol) and 3-[(4-chlorophenyl)amino]-4-methoxycyclobut-3-ene-1,2-dione (8-2) (0.094 g, 0.32 mmol) in 7 ml of dichloromethane is stirred for 16 hours at 23° C. The resulting solid is filtered using a sintered-glass filter and washed with ether. After drying, a solid is obtained in the form of a pinkish beige powder. The yield of the reaction is 64%.

¹H-NMR (δ ppm, CDCl₃): 1.85-1.93 (m, 8H); 2.27 (s, 3H); 2.63-2.69 (m, 4H); 2.8-3.0 (m, 2H); 3.17 (se, 2H); 3.42-3.49 (m, 8H); 3.86 (se, 2H); 4.69 (s, 1H); 5.53 (se, 1H); 7.19-7.38 (m, 4H)

Observed MH+=539.17; theoretical M=538.26

Melting point: 144-146° C.

### Example 9

### 3-[(4-chlorophenyl)amino]-4-({2-[(2,6-dipyrrolidin-1-ylpyrimidin-4-yl)amino]ethyl}amino)cyclobut-3-ene-1,2-dione

A mixture containing N-(2,6-dipyrrolidin-1-ylpyrimidin-4-yl)ethane-1,2-diamine as prepared in section 1-2) (0.095 g, 0.34 mmol) and 3-[(4-chlorophenyl)amino]-4-methoxycyclobut-3-ene-1,2-dione (0.082 g, 0.34 mmol) as prepared in section (8-2) in 8 ml of ethanol is stirred for 16 hours at 23° C. The solid formed is filtered using a sintered-glass filter and washed with ether. After drying, a solid is obtained in the form of a beige powder. The yield of the reaction is 35%.

¹H-NMR (δ ppm, DMSO): 1.78-1.83 (m, 8H); 3.27-3.40 (m, 10H); 3.72-3.74 (m, 2H); 4.78 (s, 1H); 6.26 (se, 1H); 7.34-7.38 (m, 4H); 7.67 (se, 1H); 9.74 (se, 1H)

Observed MH+=482.19; theoretical M=481.20

Melting point: 251-253° C.

Various salts of example 9 can be prepared, such as the hydrochloride (example 9a), sulfate (example 9b), phosphate (example 9c) and maleate salts (example 9d) described below.

### Example 9a

### 3-[(4-chlorophenyl)amino]-4-({2-[(2,6-dipyrrolidin-1-ylpyrimidin-4-yl)amino]ethyl}amino)cyclobut-3-ene-1,2-dione hydrochloride

The 3-[(4-chlorophenyl)amino]-4-({2-[(2,6-dipyrrolidin-1-ylpyrimidin-4-yl)amino]ethyl}amino)cyclobut-3-ene-1,2-dione (0.1 g, 0.21 mmol) as prepared in example 9 is stirred in 100 ml of acetone. At 23° C. a 1M solution of hydrochloric acid in ether (1 ml, 2.1 mmol) is added to this solution then stirred for 2 hours at this temperature. The solid obtained is filtered using a sintered-glass filter, washing with acetone then with water. After drying, a white powder is obtained.

¹H-NMR (δ ppm, DMSO): 1.79-1.92 (m, 8H); 3.30-3.48 (m, 10H); 3.72-3.74 (m, 2H); 5.25 (s, 1H); 7.1 (se, 1H); 7.34-7.46 (m, 4H); 8.55 (se, 1H); 10.60 (se, 1H); 10.85 (se, 1H)

Observed MH+=481.20; theoretical M=482.18

Melting point: >370° C.

### Example 9b

### 3-[(4-chlorophenyl)amino]-4-({2-[(2,6-dipyrrolidin-1-ylpyrimidin-4-yl)amino]ethyl}amino)cyclobut-3-ene-1,2-dione sulfate

The 3-[(4-chlorophenyl)amino]-4-({2-[(2,6-dipyrrolidin-1-ylpyrimidin-4-yl)amino]ethyl}amino)cyclobut-3-ene-1,2-dione (0.1 g, 0.21 mmol) as prepared in example 9 is stirred in 100 ml of acetone. At 23° C. a concentrated sulfuric acid solution (98%) (0.12 ml, 2.1 mmol) is added to this solution, then it is stirred for 10 hours at this temperature. The resulting solid is filtered using a sintered-glass filter, washing with acetone then with water. After drying, a white powder is obtained.

¹H-NMR (δ ppm, DMSO): 1.91-2.07 (m, 8H); 3.36-3.47 (m, 11H); 3.72-3.74 (m, 2H); 5.20 (s, 1H); 6.77 (se, 1H); 7.37-7.80 (m, 4H); 9.79 (se, 1H); 10-10.8 (se, 1H)

Observed MH+=481.20; theoretical M=482.16

Melting point: 263-266° C.

### Example 9c

### 3-[(4-chlorophenyl)amino]-4-({2-[(2,6-dipyrrolidin-1-ylpyrimidin-4-yl)amino]ethyl}amino)cyclobut-3-ene-1,2-dione phosphate

The 3-[(4-chlorophenyl)amino]-4-({2-[(2,6-dipyrrolidin-1-ylpyrimidin-4-yl)amino]ethyl}amino)cyclobut-3-ene-1,2-dione (0.1 g, 0.21 mmol) as prepared in example 9 is stirred in 100 ml of acetone. At 23° C. an 85% phosphoric acid solution (0.14 ml, 2.1 mmol) is added to this solution then it is stirred for 10 hours at this temperature. The resulting solid is filtered using a sintered-glass filter, washing with acetone then with water. After drying, a yellow powder is obtained.

¹H-NMR (δ ppm, DMSO): 1.83-2.07 (m, 8H); 3.32-3.40 (m, 11H); 3.72-3.74 (m, 2H); 4.91 (s, 1H); 6.82 (se, 1H); 7.37-7.44 (m, 3H); 8-8.5 (se, 1H); 10-10.5 (se, 1H)

Observed MH+=481.20; theoretical M=482.19

Melting point: 275-278° C.

### Example 9d

### 3-[(4-chlorophenyl)amino]-4-({2-[(2,6-dipyrrolidin-1-ylpyrimidin-4-yl)amino]ethyl}amino)cyclobut-3-ene-1,2-dione maleate

The 3-[(4-chlorophenyl)amino]-4-({2-[(2,6-dipyrrolidin-1-ylpyrimidin-4-yl)amino]ethyl}amino)cyclobut-3-ene-1,2-dione (0.1 g, 0.21 mmol) as prepared in example 9 is stirred in 100 ml of methanol. At 23° C. maleic acid (0.48 mg, 0.42 mmol) is added to this solution then it is stirred for 10 hours at this temperature. The resulting solid is filtered using a sintered-glass filter, washing with water. After drying, a yellow powder is obtained.

¹H-NMR (δ ppm, DMSO): 1.90 (m, 8H); 3.36-3.46 (m, 10H); 3.75 (s, 2H); 5.12 (s, 1H); 6.05 (s, 2H); 6.70 (se, 1H); 7.37-7.480 (m, 5H); 9.78 (se, 1H)

Observed MH+=481.20; theoretical M=482.17

Melting point: 248-250° C.

### Example 10

### 3-{[4-chloro-3-(trifluoromethyl)phenyl]amino}-4-({2-[(2,6-dipyrrolidin-1-ylpyrimidin-4-yl)amino]ethyl}amino)cyclobut-3-ene-1,2-dione

### 10-1) 3-{[4-chloro-3-(trifluoromethyl)phenyl]amino}-4-methoxycyclobut-3-ene-1,2-dione

The intermediate 10-1) is prepared according to the method described in section 8-2).

Observed MH+=306.19; theoretical M=305.01

### 10-2) 3-{[4-chloro-3-(trifluoromethyl)phenyl]amino}-4-({2-[(2,6-dipyrrolidin-1-ylpyrimidin-4-yl)amino]ethyl}amino)cyclobut-3-ene-1,2-dione

The compound of example 10 was synthesised according to a method analogous to the one described in example 9, using the intermediate prepared in section 1-2), by reaction with the intermediate prepared in section 10-1) above.

¹H-NMR (δ ppm, DMSO): 1.76-1.81 (m, 8H); 3.25-3.46 (m, 10H); 3.63-3.77 (m, 2H); 4.77 (s, 1H); 6.27 (se, 1H); 7.53-7.70 (m, 3H); 7.98 (s, 1H); 9.98 (se, 1H)

Observed MH+=550.25; theoretical M=549.19

Melting point: 216-218° C.

### Example 11

### 3-(biphenyl-4-ylamino)-4-({2-[(2,6-dipyrrolidin-1-ylpyrimidin-4-yl)amino]ethyl}amino)cyclobut-3-ene-1,2-dione

### 11-1) 3-(biphenyl-4-ylamino)-4-methoxycyclobut-3-ene-1,2-dione

The intermediate 11-1) is prepared according to the method described in section 8-2).

¹H-NMR (δ ppm, DMSO): 4.40 (s, 3H); 7.33-7.35 (m 1H); 7.42-7.46 (m, 4H); 7.64-7.67 (m, 4H); 10.82 (s, 1H)

Observed MH+=280.20; theoretical M=279.09

### 11-2) 3-(biphenyl-4-ylamino)-4-({2-[(2,6-dipyrrolidin-1-ylpyrimidin-4-yl)amino]ethyl}amino)cyclobut-3-ene-1,2-dione

The compound of example 11 was synthesised according to a method analogous to the one described in example 9, using the intermediate prepared in section 1-2), by reaction with the intermediate prepared in section 11-1) above.

¹H-NMR (δ ppm, DMSO): 1.78-1.82 (m, 8H); 3.27-3.41 (m, 10H); 3.74-3.75 (m, 2H); 4.80 (s, 1H); 6.26 (se, 1H); 7.32-7.64 (m, 10H); 9.74 (se, 1H)

Observed MH+=524.41; theoretical M=523.27

### Example 12

### 3-({2-[(2,6-dipyrrolidin-1-ylpyrimidin-4-yl)amino]ethyl}amino)-4-{[3-(1H-tetrazol-5-yl)phenyl]amino}cyclobut-3-ene-1,2-dione

### 12-1) 3-methoxy-4-{[4-(2H-tetrazol-5-yl)phenyl]amino}cyclobut-3-ene-1,2-dione

The intermediate 12-1) is prepared according to the method described in section 8-2).

¹H-NMR (δ ppm, DMSO): 3.1-3.3 (se, 1H); 4.39 (s, 3H); 7.57-7.61 (m 2H); 7.73-7.74 (m, 1H); 8.04 (s, 1H); 10.96 (s, 1H)

Observed MH+=272.19; theoretical M=271.07

### 12-2) 3-({2-[(2,6-dipyrrolidin-1-ylpyrimidin-4-yl)amino]ethyl}amino)-4-{[3-(1H-tetrazol-5-yl)phenyl]amino}cyclobut-3-ene-1,2-dione

The compound of example 12 was synthesised according to a method analogous to the one described in example 9, using the intermediate prepared in section 1-2), by reaction with the intermediate prepared in section 12-1) above.

¹H-NMR (δ ppm, DMSO): 1.59 (m, 8H); 3.08-3.24 (m, 11H); 3.52-3.54 (m, 2H); 4.78 (s, 1H); 6.26 (se, 1H); 7.39-7.359 (m, 5H); 9.74 (se, 1H)

Observed MH+=516.37; theoretical M=515.25

### Example 13

### N-(4-{[2-({2-[(2,6-dipyrrolidin-1-ylpyrimidin-4-yl)amino]ethyl}amino)-3,4-dioxocyclobut-1-en-1-yl]amino}phenyl)acetamide

### 13-1) N-{4-[(2-methoxy-3,4-dioxocyclobut-1-en-1-yl)amino]phenyl}acetamide

The intermediate 13-1) is prepared according to the method described in section 8-2).

¹H-NMR (δ ppm, DMSO): 2.02 (s, 3H); 4.36 (s, 3H); 7.23-7.53 (m 4H); 9.91 (s, 1H); 10.67 (s, 1H)

Observed MH+=261.22; theoretical M=260.08

### 13-2) N-(4-{[2-({2-[(2,6-dipyrrolidin-1-ylpyrimidin-4-yl)amino]ethyl}amino)-3,4-dioxocyclobut-1-en-1-yl]amino}phenyl)acetamide

The compound of example 13 was synthesised according to a method analogous to the one described in example 9, using the intermediate prepared in section 1-2), by reaction with the intermediate prepared in section 13-1) above.

¹H-NMR (δ ppm, DMSO): 1.78-1.84 (m, 8H); 2.01 (s, 3H); 3.27-3.41 (m, 10H); 3.72-3.73 (m, 2H); 4.78 (s, 1H); 6.25 (se, 1H); 7.30-7.53 (m, 5H); 9.50 (se, 1H); 9.86 (s, 1H)

Observed MH+=505.40; theoretical M=504.26

Melting point: 263-265° C.

### Example 14

### 3-({2-[(2,6-dipyrrolidin-1-ylpyrimidin-4-yl)amino]ethyl}amino)-4-[(3,4,5-trimethoxyphenyl)amino]cyclobut-3-ene-1,2-dione

### 14-1) 3-methoxy-4-[(3,4,5-trimethoxyphenyl)amino]cyclobut-3-ene-1,2-dione

The intermediate 14-1) is prepared according to the method described in section 8-2).

¹H-NMR (δ ppm, DMSO): 3.61 (s, 3H); 3.75 (s, 6H); 4.38 (s, 3H); 6.72 (s, 2H); 10.64 (s, 1H)

Observed MH+=294.22; theoretical M=293.09

### 14-2) 3-({2-[(2,6-dipyrrolidin-1-ylpyrimidin-4-yl)amino]ethyl}amino)-4-[(3,4,5-trimethoxyphenyl)amino]cyclobut-3-ene-1,2-dione

The compound of example 14 was synthesised according to a method analogous to the one described in example 9, using the intermediate prepared in section 1-2), by reaction with the intermediate prepared in section 14-1) above.

¹H-NMR (δ ppm, CDCl₃): 1.78-1.83 (m, 8H); 2-3 (me, 2H); 3.27-3.40 (m, 10H); 3.50 (se, 9H); 3.72-3.74 (m, 2H); 4.78 (s, 1H); 6.26 (se, 1H); 7.34-7.38 (m, 3H)

Observed MH+=538.28; theoretical M=537.27

Melting point: 154-156° C.

### Example 15

### 3-[(3,5-difluorophenyl)amino]-4-({2-[(2,6-dipyrrolidin-1-ylpyrimidin-4-yl)amino]ethyl}amino)cyclobut-3-ene-1,2-dione

### 15-1) 3-[(3,5-difluorophenyl)amino]-4-methoxycyclobut-3-ene-1,2-dione

The intermediate 15-1) is prepared according to the method described in section 8-2).

¹H-NMR (δ ppm, DMSO): 4.40 (s, 3H); 6.90-6.96 (t, 1H); 7.09-7.14 (m, 2H); 10.94 (s, 1H)

Observed MH+=240.23; theoretical M=239.04

### 15-2) 3-[(3,5-difluorophenyl)amino]-4-({2-[(2,6-dipyrrolidin-1-ylpyrimidin-4-yl)amino]ethyl}amino)cyclobut-3-ene-1,2-dione

The compound of example 15 was synthesised according to a method analogous to the one described in example 9, using the intermediate prepared in section 1-2), by reaction with the intermediate prepared in section 15-1) above.

¹H-NMR (δ ppm, DMSO): 1.72-1.78 (m, 8H); 3.20-3.34 (m, 10H); 3.67 (m, 2H); 4.72 (s, 1H); 6.18 (se, 1H); 6.73-7.09 (m, 3H); 7.70 (se, 1H); 9.83 (se, 1H)

Observed MH+=484.34; theoretical M=483.22

Melting point: 192-194° C.

### Example 16

### 3-[(2-chlorophenyl)amino]-4-({2-[(2,6-dipyrrolidin-1-ylpyrimidin-4-yl)amino]ethyl}amino)cyclobut-3-ene-1,2-dione

### 16-1) 3-[(2-chlorophenyl)amino]-4-methoxycyclobut-3-ene-1,2-dione

The intermediate 16-1) is prepared according to the method described in section 8-2).

Observed MH+=238.16; theoretical M=237.02

### 16-2) 3-[(2-chlorophenyl)amino]-4-({2-[(2,6-dipyrrolidin-1-ylpyrimidin-4-yl)amino]ethyl}amino)cyclobut-3-ene-1,2-dione

The compound of example 16 was synthesised according to a method analogous to the one described in example 9, using the intermediate prepared in section 1-2), by reaction with the intermediate prepared in section 16-1) above.

¹H-NMR (δ ppm, DMSO): 1.78-1.83 (m, 8H); 3.24-3.34 (m, 10H); 3.69-3.71 (m, 2H); 4.78 (se, 1H); 6.26 (se, 1H); 7.00-7.55 (m, 4H); 8.37 (se, 1H); 9.24 (se, 1H)

Observed MH+=482.34; theoretical M=481.20

Melting point: 179-181° C.

### Example 17

### 3-[(3-chlorophenyl)amino]-4-({2-[(2,6-dipyrrolidin-1-ylpyrimidin-4-yl)amino]ethyl}amino)cyclobut-3-ene-1,2-dione

### 17-1) 3-[(3-chlorophenyl)amino]-4-methoxycyclobut-3-ene-1,2-dione

The intermediate 17-1) is prepared according to the method described in section 8-2).

Observed MH+=238.16; theoretical M=237.02

### 17-2) 3-[(3-chlorophenyl)amino]-4-({2-[(2,6-dipyrrolidin-1-ylpyrimidin-4-yl)amino]ethyl}amino)cyclobut-3-ene-1,2-dione

The compound of example 17 was synthesised according to a method analogous to the one described in example 9, using the intermediate prepared in section 1-2), by reaction with the intermediate prepared in section 17-1) above.

¹H-NMR (δ ppm, DMSO): 1.77-1.82 (m, 8H); 3.26-3.44 (m, 10H); 3.72-3.74 (m, 2H); 4.77 (s, 1H); 6.24 (se, 1H); 7.02-7.69 (m, 5H); 9.74 (se, 1H)

Observed MH+=482.34; theoretical M=481.20

Melting point: 209-211° C.

### Example 18

### 3-({2-[(2,6-dipyrrolidin-1-ylpyrimidin-4-yl)amino]ethyl}amino)-4-[(4-methoxyphenyl)amino]cyclobut-3-ene-1,2-dione

### 18-1) 3-methoxy-4-[(4-methoxyphenyl)amino]cyclobut-3-ene-1,2-dione

The intermediate 18-1) is prepared according to the method described in section 8-2).

¹H-NMR (δ ppm, DMSO): 3.73 (s, 3H); 4.34 (s, 3H); 6.90-6.94 (d, 2H); 7.24 (se, 2H); 10.64 (s, 1H)

Observed MH+=234.11; theoretical M=233.07

### 18-2) 3-({2-[(2,6-dipyrrolidin-1-ylpyrimidin-4-yl)amino]ethyl}amino)-4-[(4-methoxyphenyl)amino]cyclobut-3-ene-1,2-dione

The compound of example 18 was synthesised according to a method analogous to the one described in example 9, using the intermediate prepared in section 1-2), by reaction with the intermediate prepared in section 18-1) above.

¹H-NMR (δ ppm, CDCl₃): 1.5 (se, 3H); 1.74-1.84 (m, 8H); 3.27-3.40 (m, 10H); 3.65 (s, 3H); 3.72-3.74 (m, 2H); 4.70 (s, 1H); 6.66 (m, 2H); 6.80-6.90 (m, 2H)

Observed MH+=478.23; theoretical M=477.25

Melting point: 235-237° C.

### Example 19

### 3-[(4-acetylphenyl)amino]-4-({2-[(2,6-dipyrrolidin-1-ylpyrimidin-4-yl)amino]ethyl}amino)cyclobut-3-ene-1,2-dione

### 19-1) 3-[(4-acetylphenyl)amino]-4-methoxycyclobut-3-ene-1,2-dione

The intermediate 19-1) is prepared according to the method described in section 8-2).

Observed MH+=246.24; theoretical M=245.07

### 19-2) 3-[(4-acetylphenyl)amino]-4-({2-[(2,6-dipyrrolidin-1-ylpyrimidin-4-yl)amino]ethyl}amino)cyclobut-3-ene-1,2-dione

The compound of example 19 was synthesised according to a method analogous to the one described in example 9, using the intermediate prepared in section 1-2), by reaction with the intermediate prepared in section 19-1) above.

¹H-NMR (δ ppm, DMSO): 1.88-1.97 (m, 8H); 3.23-3.50 (m, 13H); 3.81-3.82 (m, 2H); 4.94 (s, 1H); 6.52 (se, 1H); 7.56-7.58 (m, 2H); 7.97-8.01 (m, 3H); 10.19 (se, 1H)

Observed MH+=490.13; theoretical M=489.25

Melting point: 186-188° C.

### Example 20

### 3-({2-[(2,6-dipyrrolidin-1-ylpyrimidin-4-yl)amino]ethyl}amino)-4-[(3-methoxyphenyl)amino]cyclobut-3-ene-1,2-dione

### 20-1) 3-methoxy-4-[(3-methoxyphenyl)amino]cyclobut-3-ene-1,2-dione

The intermediate 20-1) is prepared according to the method described in section 8-2).

Observed MH+=234.20; theoretical M=233.07

### 20-2) 3-({2-[(2,6-dipyrrolidin-1-ylpyrimidin-4-yl)amino]ethyl}amino)-4-[(3-methoxyphenyl)amino]cyclobut-3-ene-1,2-dione

The compound of example 20 was synthesised according to a method analogous to the one described in example 9, using the intermediate prepared in section 1-2), by reaction with the intermediate prepared in section 20-1) above.

¹H-NMR (δ ppm, DMSO): 1.72-1.78 (m, 8H); 3.21-3.34 (m, 10H); 3.68 (s, 5H); 4.73 (s, 1H); 6.20-7.63 (m, 6H); 9.57 (se, 1H)

Observed MH+=478.20; theoretical M=477.25

Melting point: 205-207° C.

### Example 21

### tert-butyl (4-{[2-({2-[(2,6-dipyrrolidin-1-ylpyrimidin-4-yl)amino]ethyl}amino)-3,4-dioxocyclobut-1-en-1-yl]amino}benzyl)methylcarbamate

### 21-1) tert-butyl {4-[(2-methoxy-3,4-dioxocyclobut-1-en-1-yl)amino]benzyl}methylcarbamate

The intermediate 21-1) is prepared according to the method described in section 8-2).

¹H-NMR (δ ppm, CDCl₃): 1.41 (s, 9H); 2.74 (s, 3H); 4.30 (s, 2H); 4.43 (s, 3H); 7.17-7.19 (d, 4H); 7.80 (se, 1H)

Observed MH+=347.24; theoretical M=346.15

### 21-2) tert-butyl(4-{[2-({2-[(2,6-dipyrrolidin-1-ylpyrimidin-4-yl)amino]ethyl}amino)-3,4-dioxocyclobut-1-en-1-yl]amino}benzyl)methylcarbamate

The compound of example 21 was synthesised according to a method analogous to the one described in example 9, using the intermediate prepared in section 1-2), by reaction with the intermediate prepared in section 21-1) above.

¹H-NMR (δ ppm, CDCl₃): 1.38 (s, 9H); 1.73-1.85 (m, 8H); 2.0-2.2 (me, 2H); 2.70 (s, 3H); 3.32-3.34 (m, 8H); 3.47 (m, 2H); 3.81 (m, 2H); 4.25 (s, 2H); 4.70 (s, 1H); 7.05-7.20 (m, 4H)

Observed MH+=591.26; theoretical M=590.33

### Example 22

### 3-({2-[(2,6-dipyrrolidin-1-ylpyrimidin-4-yl)amino]ethyl}amino)-4-({4-[(methylamino)methyl]phenyl}amino)cyclobut-3-ene-1,2-dione hydrochloride

A 1N solution of hydrochloric acid diluted in ether (0.7 ml, 0.66 mmol) is added to a solution containing the compound tert-butyl (4-{[2-({2-[(2,6-dipyrrolidin-1-ylpyrimidin-4-yl)amino]ethyl}amino)-3,4-dioxocyclobut-1-en-1-yl]amino}benzyl)methylcarbamate (0.05 g, 0.12 mmol) isolated in example 21 in 5 ml of ethanol. It is stirred for one hour at room temperature then the solvent and excess acid are eliminated using a rotavapor. Ether is added then the resulting solid is filtered.

¹H-NMR (δ ppm, DMSO): 1.77-1.93 (m, 8H); 2.51 (s, 3H); 3.38-3.48 (m, 10H); 3.72 (se, 2H); 4.02 (s, 2H); 5.23 (s, 1H); 7.30-7.55 (m, 5H); 8.83 (m, 2H); 11.94 (se, 1H)

Observed MH+=491.31; theoretical M=490.28

Melting point: >260° C.

### Example 23

### 3-[(4-cyclohexylphenyl)amino]-4-({2-[(2,6-dipyrrolidin-1-ylpyrimidin-4-yl)amino]ethyl}amino)cyclobut-3-ene-1,2-dione

### 23-1) 3-[(4-cyclohexylphenyl)amino]-4-methoxycyclobut-3-ene-1,2-dione

The intermediate 23-1) is prepared according to the method described in section 8-2).

¹H-NMR (δ ppm, CDCl₃): 1.16-1.34 (m, 5H); 1.67-1.80 (m, 5H); 2.40-2.42 (q, 1H); 4.43 (s, 3H); 7.13 (s, 4H); 7.80 (se, 1H)

Observed MH+=286.18; theoretical M=285.14

### 23-2) 3-[(4-cyclohexylphenyl)amino]-4-({2-[(2,6-dipyrrolidin-1-ylpyrimidin-4-yl)amino]ethyl}amino)cyclobut-3-ene-1,2-dione

The compound of example 23 was synthesised according to a method analogous to the one described in example 9, using the intermediate prepared in section 1-2), by reaction with the intermediate prepared in section 23-1) above.

¹H-NMR (δ ppm, CDCl₃): 1.17-1.29 (m, 8H); 1.68-1.83 (m, 13H); 2.30 (m, 1H); 3.31-3.46 (m, 10H); 3.82 (s, 2H); 4.69 (s, 1H); 7.00-7.19 (m, 4H)

Observed MH+=530.29; theoretical M=529.32

Melting point: 218-220° C.

### Example 24

### 3-({2-[(2,6-dipyrrolidin-1-ylpyrimidin-4-yl)amino]ethyl}amino)-4-[(4-propylphenyl)amino]cyclobut-3-ene-1,2-dione

### 24-1) 3-methoxy-4-[(4-propylphenyl)amino]cyclobut-3-ene-1,2-dione

The intermediate 24-1) is prepared according to the method described in section 8-2).

¹H-NMR (δ ppm, CDCl₃): 0.87 (t, 3H); 1.51-1.61 (m, 2H); 2.48-2.52 (t, 2H); 4.42 (s, 3H); 7.09-7.14 (m, 4H); 7.70-7.90 (se, 1H)

Observed MH+=246.15; theoretical M=245.11

### 24-2) 3-({2-[(2,6-dipyrrolidin-1-ylpyrimidin-4-yl)amino]ethyl}amino)-4-[(4-propylphenyl)amino]cyclobut-3-ene-1,2-dione

The compound of example 24 was synthesised according to a method analogous to the one described in example 9, using the intermediate prepared in section 1-2), by reaction with the intermediate prepared in section 24-1) above.

¹H-NMR (δ ppm, CDCl₃): 0.85 (t, 3H); 1.47-1.53 (m, 2H); 1.73-1.85 (m, 11H); 2.42 (t, 2H); 3.31-3.48 (m, 10H); 3.82 (m, 2H); 4.69 (s, 1H); 6.94-6.98 (m, 4H)

Observed MH+=490.27; theoretical M=489.29

### Example 25

### tert-butyl (4-{[2-({2-[(2,6-dipyrrolidin-1-ylpyrimidin-4-yl)amino]ethyl}amino)-3,4-dioxocyclobut-1-en-1-yl]amino}benzyl)carbamate

### 25-1) tert-butyl {4-[(2-methoxy-3,4-dioxocyclobut-1-en-1-yl)amino]benzyl}carbamate

The intermediate 25-1) is prepared according to the method described in section 8-2).

Observed MH+=333.18; theoretical M=332.14

### 25-2) tert-butyl (4-{[2-({2-[(2,6-dipyrrolidin-1-ylpyrimidin-4-yl)amino]ethyl}amino)-3,4-dioxocyclobut-1-en-1-yl]amino}benzyl)carbamate

The compound of example 25 was synthesised according to a method analogous to the one described in example 9, using the intermediate prepared in section 1-2), by reaction with the intermediate prepared in section 25-1) above.

¹H-NMR (δ ppm, DMSO): 1.32 (s, 9H); 1.71-1.79 (m, 8H); 3.21-3.36 (m, 10H); 3.66 (m, 2H); 3.99 (m, 2H); 4.2 (m, 1H); 4.73 (s, 1H); 6.20 (se, 1H); 7.09-7.26 (m, 4H); 7.70 (se, 1H); 9.60 (se, 1H)

Observed MH+=577.25; theoretical M=576.32

Melting point: 176-180° C.

### Example 26

### 3-(1,3-benzodioxol-5-ylamino)-4-({2-[(2,6-dipyrrolidin-1-ylpyrimidin-4-yl)amino]ethyl}amino)cyclobut-3-ene-1,2-dione

### 26-1) 3-(1,3-benzodioxol-5-ylamino)-4-methoxycyclobut-3-ene-1,2-dione

The intermediate 26-1) is prepared according to the method described in section 8-2).

Observed MH+=248.10; theoretical M=247.05

### 26-2) 3-(1,3-benzodioxol-5-ylamino)-4-({2-[(2,6-dipyrrolidin-1-ylpyrimidin-4-yl)amino]ethyl}amino)cyclobut-3-ene-1,2-dione

The compound of example 26 was synthesised according to a method analogous to the one described in example 9, using the intermediate prepared in section 1-2), by reaction with the intermediate prepared in section 26-1) above.

¹H-NMR (δ ppm, DMSO): 1.77-1.84 (m, 8H); 3.27-3.40 (m, 10H); 3.71 (m, 2H); 4.78 (s, 1H); 5.93 (s, 2H); 6.25 (m, 1H); 6.67-7.20 (m, 3H); 7.70 (se, 1H); 9.60 (se, 1H)

Observed MH+=492.22; theoretical M=491.23

Melting point: 180-190° C.

### Example 27

### 3-({2-[(2,6-dipyrrolidin-1-ylpyrimidin-4-yl)amino]ethyl}amino)-4-[(4-hydroxyphenyl)amino]cyclobut-3-ene-1,2-dione

### 27-1) 3-[(4-hydroxyphenyl)amino]-4-methoxycyclobut-3-ene-1,2-dione

The intermediate 27-1) is prepared according to the method described in section 8-2).

¹H-NMR (δ ppm, DMSO): 4.33 (s, 3H); 6.70-6.74 (d, 2H); 7.12 (se, 2H); 9.36 (se, 1H); 10.52 (se, 1H)

Observed MH+=220.10; theoretical M=219.05

### 27-2) 3-({2-[(2,6-dipyrrolidin-1-ylpyrimidin-4-yl)amino]ethyl}amino)-4-[(4-hydroxyphenyl)amino]cyclobut-3-ene-1,2-dione

The compound of example 27 was synthesised according to a method analogous to the one described in example 9, using the intermediate prepared in section 1-2), by reaction with the intermediate prepared in section 27-1) above.

¹H-NMR (δ ppm, DMSO): 1.78-1.84 (m, 8H); 3.27-3.46 (m, 10H); 3.71-3.73 (m, 2H); 4.78 (s, 1H); 6.25 (m, 1H); 6.68-6.73 (m, 2H); 7.16-7.18 (m, 2H); 7.70 (se, 1H); 9.60 (se, 2H)

Observed MH+=464.22; theoretical M=463.23

Melting point: >260° C.

### Example 28

### 3-{[4-(aminomethyl)phenyl]amino}-4-({2-[(2,6-dipyrrolidin-1-ylpyrimidin-4-yl)amino]ethyl}amino)cyclobut-3-ene-1,2-dione hydrochloride

The compound is synthesised from example 25 according to a method described in example 22.

¹H-NMR (δ ppm, DMSO): 1.73-1.87 (m, 8H); 3.27-3.46 (m, 8H); 3.64-3.67 (m, 2H); 3.86-3.90 (m, 2H); 5.717 (s, 1H); 6.25 (m, 1H); 7.35-7.49 (m, 4H); 8.20 (se, 3H); 8.98 (se, 1H); 11.01-11.15 (m, 2H)

Observed MH+=477.21; theoretical M=476.26

Melting point: 224-226° C.

### Example 29

### tert-butyl [2-(4-{[2-({2-[(2,6-dipyrrolidin-1-ylpyrimidin-4-yl)amino]ethyl}amino)-3,4-dioxocyclobut-1-en-1-yl]amino}phenyl)ethyl]carbamate

### 29-1) tert-butyl (2-{4-[(2-methoxy-3,4-dioxocyclobut-1-en-1-yl)amino]phenyl}ethyl)carbamate

The intermediate 29-1) is prepared according to the method described in section 8-2).

Observed MH+=347.19; theoretical M=346.15

### 29-2) tert-butyl [2-(4-{[2-[(2,6-dipyrrolidin-1-ylpyrimidin-4-yl)amino]ethyl}amino)-3,4-dioxocyclobut-1-en-1-yl]amino}phenyl)ethyl]carbamate

The compound of example 29 was synthesised according to a method analogous to the one described in example 9, using the intermediate prepared in section 1-2), by reaction with the intermediate prepared in section 29-1) above.

¹H-NMR (δ ppm, DMSO): 1.31 (s, 9H); 1.73-1.78 (m, 8H); 2.55-2.59 (m, 2H); 3.02-3.05 (m, 2H); 3.21-3.34 (m, 10H); 3.67-3.69 (m, 2H); 4.74 (s, 1H); 6.25 (se, 1H); 6.77 (se, 1H); 7.06 (m, 2H); 7.24 (m, 2H); 7.80 (se, 1H); 9.7 (se, 1H)

Observed MH+=591.22; theoretical M=590.33

Melting point: 216-218° C.

### Example 30

### 3-{[4-(2-aminoethyl)phenyl]amino}-4-({2-[(2,6-dipyrrolidin-1-ylpyrimidin-4-yl)amino]ethyl}amino)cyclobut-3-ene-1,2-dione hydrochloride

The compound is synthesised from example 29 according to a method described in example 22.

¹H-NMR (δ ppm, DMSO): 1.72-1.86 (m, 8H); 2.74-2.78 (m, 2H); 2.91-2.95 (m, 2H); 3.35-3.40 (m, 11H); 3.63-3.66 (m, 2H); 5.19 (s, 1H); 7.13-7.15 (m, 2H); 7.40-7.45 (m, 2H); 7.88 (se, 3H); 8.80 (se, 1H); 10.8 (m, 2H)

Observed MH+=491.22; theoretical M=490.28

Melting point: 250-252° C.

### Example 31

### 3-({2-[(2,6-dipyrrolidin-1-ylpyrimidin-4-yl)amino]ethyl}amino)-4-[(4-fluorophenyl)amino]cyclobut-3-ene-1,2-dione

### 31-1) 3-[(4-fluorophenyl)amino]-4-methoxycyclobut-3-ene-1,2-dione

The intermediate 31-1) is prepared according to the method described in section 8-2).

¹H-NMR (δ ppm, DMSO): 4.36 (s, 3H); 7.17-7.35 (m, 4H); 10.73 (se, 1H)

Observed MH+=222.10; theoretical M=221.05

### 31-2) 3-({2-[(2,6-dipyrrolidin-1-ylpyrimidin-4-yl)amino]ethyl}amino)-4-[(4-fluorophenyl)amino]cyclobut-3-ene-1,2-dione

The compound of example 31 was synthesised according to a method analogous to the one described in example 9, using the intermediate prepared in section 1-2), by reaction with the intermediate prepared in section 31-1) above.

¹H-NMR (δ ppm, DMSO): 1.78-1.84 (m, 8H); 3.26-3.40 (m, 10H); 3.71-3.73 (m, 2H); 4.81 (se, 1H); 6.25 (se, 1H); 7.13-7.40 (m, 4H); 7.80 (se, 1H); 9.80 (se, 1H)

Observed MH+=466.21; theoretical M=465.23

Melting point: 241-243° C.

### Example 32

### 3-({2-[(2,6-dipyrrolidin-1-ylpyrimidin-4-yl)amino]ethyl}amino)-4-{[4-(trifluoromethyl)phenyl]amino}cyclobut-3-ene-1,2-dione

### 32-1) 3-methoxy-4-{[4-(trifluoromethyl)phenyl]amino}cyclobut-3-ene-1,2-dione

The intermediate 32-1) is prepared according to the method described in section 8-2).

¹H-NMR (δ ppm, DMSO): 4.40 (s, 3H); 7.54-7.56 (d, 2H); 7.70-7.72 (d, 2H); 10.99 (s, 1H)

Observed MH+=272.03; theoretical M=271.05

### 32-2) 3-({2-[(2,6-dipyrrolidin-1-ylpyrimidin-4-yl)amino]ethyl}amino)-4-{[4-(trifluoromethyl)phenyl]amino}cyclobut-3-ene-1,2-dione

The compound of example 32 was synthesised according to a method analogous to the one described in example 9, using the intermediate prepared in section 1-2), by reaction with the intermediate prepared in section 32-1) above.

¹H-NMR (δ ppm, DMSO): 1.77-1.84 (m, 8H); 3.27-3.41 (m, 10H); 3.71-3.73 (m, 2H); 4.79 (s, 1H); 6.25 (se, 1H); 7.53-7.83 (m, 5H); 9.94 (se, 1H)

Observed MH+=516.20; theoretical M=515.23

Melting point: >250° C.

### Example 33

### 3-({2-[(2,6-dipyrrolidin-1-ylpyrimidin-4-yl)amino]ethyl}amino)-4-[(4-cyanophenyl)amino]cyclobut-3-ene-1,2-dione

### 33-1) 4-[(2-methoxy-3,4-dioxocyclobut-1-en-1-yl)amino]benzonitrile

The intermediate 33-1) is prepared according to the method described in section 8-2).

¹H-NMR (δ ppm, DMSO): 4.40 (s, 3H); 7.54-7.56 (d, 2H); 7.79-7.82 (d, 2H); 11.05 (s, 1H)

Observed MH+=229.12; theoretical M=228.05

### 33-2) 3-({2-[(2,6-dipyrrolidin-1-ylpyrimidin-4-yl)amino]ethyl}amino)-4-[(4-cyanophenyl)amino]cyclobut-3-ene-1,2-dione

The compound of example 33 was synthesised according to a method analogous to the one described in example 9, using the intermediate prepared in section 1-2), by reaction with the intermediate prepared in section 33-1) above.

¹H-NMR (δ ppm, DMSO): 1.78-1.84 (m, 8H); 3.27-3.44 (m, 10H); 3.71-3.73 (m, 2H); 4.80 (s, 1H); 6.29 (se, 1H); 7.52-7.91 (m, 5H); 10.01 (se, 1H)

Observed MH+=473.21; theoretical M=472.23

Melting point: >250° C.

### Example 34

### 3-({2-[(2,6-dipyrrolidin-1-ylpyrimidin-4-yl)amino]ethyl}amino)-4-[(4-nitrophenyl)amino]cyclobut-3-ene-1,2-dione

### 34-1) 3-methoxy-4-[(4-nitrophenyl)amino]cyclobut-3-ene-1,2-dione

The intermediate 34-1) is prepared according to the method described in section 8-2).

¹H-NMR (δ ppm, DMSO): 4.41 (s, 3H); 7.56-7.59 (d, 2H); 8.21-8.23 (d, 2H); 11.21 (s, 1H)

Observed MH+=249.05; theoretical M=248.04

### 34-2) 3-({2-[(2,6-dipyrrolidin-1-ylpyrimidin-4-yl)amino]ethyl}amino)-4-[(4-nitrophenyl)amino]cyclobut-3-ene-1,2-dione

The compound of example 34 was synthesised according to a method analogous to the one described in example 9, using the intermediate prepared in section 1-2), by reaction with the intermediate prepared in section 34-1) above.

¹H-NMR (δ ppm, DMSO): 1.83-1.89 (m, 8H); 3.33-3.49 (m, 10H); 3.81-3.83 (m, 2H); 4.85 (s, 1H); 6.34 (se, 1H); 7.59-8.26 (m, 5H); 10.40 (se, 1H)

Observed MH+=493.22; theoretical M=492.22

### Example 35

### 3-anilino-4-({2-[(2,6-dipyrrolidin-1-ylpyrimidin-4-yl)amino]ethyl}amino)cyclobut-3-ene-1,2-dione

### 35-1) 3-methoxy-4-(phenylamino)cyclobut-3-ene-1,2-dione

The intermediate 35-1) is prepared according to the method described in section 8-2).

¹H-NMR (δ ppm, DMSO): 4.37 (s, 3H); 7.08-7.7.12 (m, 1H); 7.34-7.35 (m, 4H); 10.72 (se, 1H)

Observed MH+=204.14; theoretical M=203.06

### 35-2) 3-anilino-4-({2-[(2,6-dipyrrolidin-1-ylpyrimidin-4-yl)amino]ethyl}amino)cyclobut-3-ene-1,2-dione

The compound of example 35 was synthesised according to a method analogous to the one described in example 9, using the intermediate prepared in section 1-2), by reaction with the intermediate prepared in section 35-1) above.

¹H-NMR (δ ppm, DMSO): 1.77-1.84 (m, 8H); 3.27-3.42 (m, 10H); 3.72-3.74 (m, 2H); 4.79 (s, 1H); 6.26 (se, 1H); 6.98-7.41 (m, 5H); 7.80 (se, 1H); 9.80 (se, 1H)

Observed MH+=448.20; theoretical M=447.24

### Example 36

### 3-[(4-chloro-3-fluorophenyl)amino]-4-({2-[(2,6-dipyrrolidin-1-ylpyrimidin-4-yl)amino]ethyl}amino)cyclobut-3-ene-1,2-dione

### 36-1) 3-[(4-chloro-3-fluorophenyl)amino]-4-methoxycyclobut-3-ene-1,2-dione

The intermediate 36-1) is prepared according to the method described in section 8-2).

¹H-NMR (δ ppm, DMSO): 4.39 (s, 3H); 7.19-7.22 (m, 1H); 7.43-7.46 (dd, 1H); 7.53-7.57 (m, 1H); 10.92 (s, 1H)

Observed MH+=256.07; theoretical M=255.01

### 36-2) 3-[(4-chloro-3-fluorophenyl)amino]-4-({2-[(2,6-dipyrrolidin-1-ylpyrimidin-4-yl)amino]ethyl}amino)cyclobut-3-ene-1,2-dione

The compound of example 36 was synthesised according to a method analogous to the one described in example 9, using the intermediate prepared in section 1-2), by reaction with the intermediate prepared in section 36-1) above.

¹H-NMR (δ ppm, DMSO): 1.78-1.84 (m, 8H); 3.27-3.44 (m, 10H); 3.71-3.73 (m, 2H); 4.80 (s, 1H); 6.29 (se, 1H); 7.52-7.91 (m, 4H); 10.00 (se, 1H)

Observed MH+=500.14; theoretical M=499.19

### Example 37

### 3-({2-[(2,6-dipyrrolidin-1-ylpyrimidin-4-yl)amino]ethyl}amino)-4-(pyridin-3-ylamino)cyclobut-3-ene-1,2-dione

### 37-1) 3-methoxy-4-(pyridin-3-ylamino)cyclobut-3-ene-1,2-dione

The intermediate 37-1) is prepared according to the method described in section 8-2).

¹H-NMR (δ ppm, DMSO): 4.41 (s, 3H); 7.56-7.59 (d, 2H); 8.21-8.23 (d, 2H); 11.21 (s, 1H)

Observed MH+=205.14; theoretical M=204.05

### 37-2) 3-({2-[(2,6-dipyrrolidin-1-ylpyrimidin-4-yl)amino]ethyl}amino)-4-(pyridin-3-ylamino)cyclobut-3-ene-1,2-dione

The compound of example 37 was synthesised according to a method analogous to the one described in example 9, using the intermediate prepared in section 1-2), by reaction with the intermediate prepared in section 37-1) above.

Observed MH+=449.21; theoretical M=448.23

### Example 38

### 3-[(4-chlorophenyl)amino]-4-[(2-{[2-(diethylamino)-6-pyrrolidin-1-ylpyrimidin-4-yl]amino}ethyl)amino]cyclobut-3-ene-1,2-dione

### 38-1) 2,4-dichloro-6-pyrrolidin-1-ylpyrimidine

The pyrrolidine compound (0.956 ml, 12 mmol) diluted in 8 ml of tetrahydrofuran is added to a solution containing 2,4,6-trichloropyrimidine (2 g, 12 mmol) and triethylamine (1.95 ml, 14 mmol) in 10 ml of tetrahydrofuran at a temperature of 0° C. It is stirred for 0.5 hour at this temperature then for 5 hours at 23° C. Next 50 ml of water is added and it is then extracted with 2×30 ml of ethyl acetate. The organic phase is dried over sodium sulfate and the solvent is then eliminated using a rotary evaporator. The resulting oil is applied to a Biotage type chromatography column containing silica (eluent: ethyl acetate-heptane: 15-85 to 25-75) and a solid is obtained in the form of a white powder. The yield of the reaction is 60%.

¹H-NMR (δ ppm, DMSO): 1.85-1.99 (m, 4H); 3.34-3.49 (m, 4H); 6.64 (s, 1H)

Observed MH+=218.00; theoretical M=217.02

### 38-2) 4-chloro-N,N-diethyl-6-pyrrolidin-1-ylpyrimidin-2-amine

The diethylamine compound (0.5 ml, 7 mmol) is added to a solution containing 2,4-dichloro-6-pyrrolidin-1-ylpyrimidine (1.5 g, 7 mmol) and triethylamine (1.15 ml, 8 mmol) in 60 ml of tetrahydrofuran at a temperature of 23° C. It is heated for 2 hours at 60° C. then 0.3 ml of triethylamine is added and it is stirred for 10 hours at 23° C. Next 50 ml of water is added and it is then extracted with 2×30 ml of ethyl acetate. The organic phase is dried over sodium sulfate and the solvent is then eliminated using a rotary evaporator. The resulting oil is applied to a Biotage type chromatography column containing silica (eluent: ethyl acetate-heptane: 1-4) and a solid is obtained in the form of a white powder. The yield of the reaction is 21%.

¹H-NMR (δ ppm, DMSO): 1.08 (t, 6H); 1.88 (m, 4H); 3.27-3.50 (m, 8H); 5.73 (s, 1H)

Observed MH+=255.17; theoretical M=254.13

### 38-3) N⁴-(2-aminoethyl)-N²,N²-diethyl-6-pyrrolidin-1-ylpyrimidine-2,4-diamine

In a sealed glass tube suitable for microwave heating, the compound 4-chloro-N,N-diethyl-6-pyrrolidin-1-ylpyrimidin-2-amine as prepared in section 38-2) (0.36 g, 1.4 mmol) and ethylenediamine (0.76 ml, 11 mmol) are heated in a microwave oven (Biotage, Emrys Optimizer) at 190° C. for 3600 seconds. When the reaction is complete, 20 ml of water are added and the reaction mixture is then extracted with ethyl acetate. It is washed with 3×20 ml of water then the organic phase is dried over sodium sulfate. It is evaporated to dryness until a brown oil is obtained. The yield of the reaction is 80%.

Observed MH+=279.19; theoretical M=278.20

### 38-4) 3-[(4-chlorophenyl)amino]-4-[(2-{[2-(diethylamino)-6-pyrrolidin-1-ylpyrimidin-4-yl]amino}ethyl)amino]cyclobut-3-ene-1,2-dione

A mixture containing N⁴-(2-aminoethyl)-N²,N²-diethyl-6-pyrrolidin-1-ylpyrimidine-2,4-diamine as prepared in section 38-3) (0.31 g, 1.1 mmol) and 3-[(4-chlorophenyl)amino]-4-methoxycyclobut-3-ene-1,2-dione (0.27 g, 1.1 mmol) as prepared in section 8-2) in 8 ml of methanol is heated at 60° C. for 2 hours. When the reaction is complete, the resulting solid is filtered using a sintered-glass filter and washed with methanol. After drying, a solid is obtained in the form of an orange powder.

Observed MH+=484.20; theoretical M=483.21

### Pharmacological Study of the Compounds of the Invention:

### Test Protocols

### i) Determination of the Phosphatase Activity of the Purified Recombinant CDC25C Enzyme:

The phosphatase activity of the MBP-CDC25C protein is evaluated through its dephosphorylation of 3-O-methylfluorescein-phosphate (OMFP) to form 3-O-methylfluorescein (OMF), determining the fluorescence of the reaction product at 475 nm. This assay can be used to identify inhibitors of the recombinant CDC25 enzyme. The preparation of the MBP-CDC25C fusion protein is described in the PCT patent application published under the number WO 01/44467.

The reaction is performed in 384-well plates in a final volume of 50 μl. The MBP-CDC25C protein (prepared as described hereinabove) is stored in the following elution buffer: 20 mM Tris-HCl pH 7.4; 250 mM NaCl; 1 mM EDTA; 1 mM dithiothreitol (DTT); 10 mM maltose. It is diluted to a concentration of 60 μM in the following reaction buffer: 50 mM Tris-HCl pH 8.2; 50 mM NaCl; 1 mM DTT; 20% glycerol. The background noise is determined using with the buffer without adding the enzyme. The products are tested at decreasing concentrations from 40 μM. The reaction is initiated by the addition of OMFP solution to a final concentration of 500 μM (prepared immediately before use from a 12.5 mM stock solution in 100% DMSO (Sigma #M2629)). After 4 hours at 30° C. in a disposable 384-well plate, the fluorescence measured at OD 475 nm is read on a Victor² plate reader (EGG-Wallac). The concentration that produces 50% inhibition of the enzyme reaction is calculated from three independent experiments. Only the values that fall within the linear part of the sigmoid curve are used for the linear regression analysis.

### ii) Characterisation of Anti-Proliferative Activity:

By way of an example, the effect of treating two human cell lines MIA PaCa-2 and DU 145 with the compounds of the examples described hereinbefore will be studied. The cell lines DU 145 (human prostate carcinoma cells) and MIA PaCa-2 (human pancreatic carcinoma cells) were obtained from the American Tissue Culture Collection (Rockville, Md., USA). A 96-well plate was inoculated on day 0 with cells in 80 μl of Dulbecco's modified Eagle's medium (Gibco-Brl, Cergy-Pontoise, France) with 10% heat-inactivated foetal calf serum (Gibco-Brl, Cergy-Pontoise, France), 50,000 units/l of penicillin and 50 mg/l of streptomycin (Gibco-Brl, Cergy-Pontoise, France) and 2 mM of glutamine (Gibco-Brl, Cergy-Pontoise, France). The cells were treated on day 1 for 96 hours with increasing concentrations of each test compound up to 10 μM. At the end of this period, cell proliferation is quantified by means of a colorimetric test based on cleavage of the tetrazolium salt WST1 by the mitochondrial dehydrogenases in the viable cells, resulting in formation of formazan (Boehringer Mannheim, Meylan, France). These tests are performed in duplicate with 8 determinations per concentration tested. For each test compound, the values within the linear part of the sigmoid curve were subjected to linear regression analysis and used to estimate the IC₅₀ inhibitory concentration. The products are solubilised in dimethylsulfoxide (DMSO) at a concentration of 10⁻² M and used in culture with a final DMSO concentration of 0.1%.

### Results of the Tests:

a) The IC₅₀ of the compounds of the following examples for the phosphatase activity of the purified recombinant CDC25-C enzyme is less than or equal to:
   15,000 nM: examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36.
   5000 nM: examples 1, 2, 3, 4, 5, 6, 7, 8, 9, 11, 12, 13, 14, 15, 16, 17, 18, 19, 20, 21, 22, 23, 24, 25, 26, 28, 29, 30, 31, 32, 33, 34, 35, 36
   1000 nM: examples 3, 5, 9, 15, 16, 18, 19, 21, 24, 25, 26, 29, 31, 32, 33, 34, 35, 36.
b) The IC₅₀ of the compounds of the following examples for proliferation of the MIA PaCa-2 cell lines is less than or equal to:
   10,000 nM: examples: 5, 8, 9, 10, 11, 14, 15, 16, 17, 18, 19, 20, 21, 22, 23, 24, 25, 26, 28, 29, 31, 32, 33, 34, 35, 36;
   2000 nM: examples: 9, 11, 15, 32, 34, 36.
c) The IC₅₀ of the compounds of the following examples for proliferation of the DU 145 cell lines is less than or equal to:
   10,000 nM: examples: 5, 8, 9, 10, 11, 15, 17, 18, 19, 20, 21, 23, 24, 25, 26, 29, 31, 32, 33, 34, 36;
   5000 nM: examples: 9, 10, 11, 15, 17, 24, 32, 34, 36;
   2000 nM: example: 9.

1. A compound of formula (I) 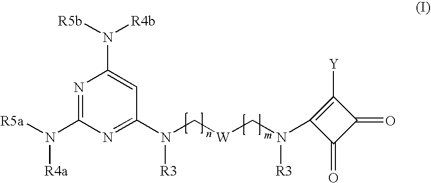
in a racemic form, an enantiomeric form or any combination thereof, where:
Y represents independently an NR₁R₂ or OR₁₃ group;
W represents independently —NR₆— or —CR₆R₇—;
each R3 independently represents a hydrogen atom or an alkyl group;
n and m are integers from 0 to 4 inclusive;
R4a and R5a represent independently a hydrogen atom, an alkyl group or alternatively together with the nitrogen atom to which they are attached form a heterocycloalkyl;
R4b and R5b represent independently a hydrogen atom, an alkyl group or alternatively together with the nitrogen atom to which they are attached form a heterocycloalkyl;
R₁, R₂ and R₁₃ represent independently a hydrogen atom or a group selected from:
alkyl;
arylalkyl optionally substituted by one or more identical or different halo groups;
heteroaryl optionally substituted by one or more identical or different groups selected from: halo, hydroxy, cyano, nitro, alkyl, alkoxy, haloalkyl, or haloalkoxy;
aryl optionally substituted by one or more identical or different groups selected from: halo, hydroxy, cyano, nitro, alkyl, alkoxy, haloalkyl, haloalkoxy, aminoalkyl, alkylaminoalkyl, dialkylaminoalkyl, cycloalkyl, aryl, heteroaryl, —NH—(C═O)—R14, —(C═O)—R14 or —(CH2)ₚ-NR14-(C═O)—O—R15 where p represents an integer between 1 and 3 inclusive; or
a group of formula 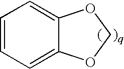
where q represents 1 or 2;
or alternatively R₁ and R₂ together with the nitrogen atom to which they are attached form a heterocycloalkyl;
R₆ and R₇ represent independently a hydrogen atom or an alkyl group;
R₁₄ and R₁₅ represent independently a hydrogen atom or an alkyl group;
or a pharmaceutically acceptable salt of the compound.

2. The compound according to claim 1, wherein R4a, R5a, R4b and R5b are such that the groups —NR4aR5a and —NR4bR5b are identical.

3. The compound according to claim 1, wherein R4a, R5a, R4b and R5b are such that the groups —NR4aR5a and —NR4bR5b are different.

4. The compound according to claim 1, wherein:
Y represents independently an NR₁R₂ or OR₁₃ group;
W represents independently —NR₆— or —CR₆R₇—;
R3 represents a hydrogen atom;
n and m are integers from 0 to 2 inclusive;
R4a and R5a represent an alkyl group or alternatively together with the nitrogen atom to which they are attached form a heterocycloalkyl;
R4b and R5b represent an alkyl group or alternatively together with the nitrogen atom to which they are attached form a heterocycloalkyl;
R₁ and R₂ represent independently a hydrogen atom, or a group selected from:
alkyl;
arylalkyl optionally substituted by one or more identical or different halo groups;
heteroaryl;
aryl optionally substituted by one or more identical or different groups selected from: halo, hydroxy, cyano, nitro, alkyl, alkoxy, haloalkyl, aminoalkyl, alkylaminoalkyl, dialkylaminoalkyl, cycloalkyl, aryl, heteroaryl, —NH—(C═O)—R14, —(C═O)—R14 or —(CH2)ₚ-NR14-(C═O)—O—R15 where p represents an integer between 1 and 2 inclusive; or
a group of formula 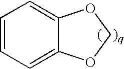
where q represents 1 or 2;
or alternatively R₁ and R₂ together with the nitrogen atom to which they are attached form a heterocycloalkyl;
R₆ and R₇ represent independently a hydrogen atom or an alkyl group;
R₁₃ represents a hydrogen atom or an alkyl group;
R₁₄ and R₁₅ represent independently a hydrogen atom or an alkyl group.

5. The compound according to claim 1, wherein
Y represents an NR₁R₂ group;
W represents —CR₆R₇—;
R₁ and R₂ represent independently a hydrogen atom, or a group selected from:
alkyl,
arylalkyl optionally substituted by a halogen atom;
aryl optionally substituted by one or more identical or different groups selected from: halo, cyano, nitro, alkyl, alkoxy, haloalkyl, aryl, —(C═O)—R₁₄ or —(CH₂)ₚ—NR₁₄—(C═O)—O—R₁₅ where p represents an integer between 1 and 2 inclusive; or
a group of formula 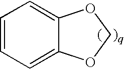
where q represents 1;
R₆ and R₇ represent a hydrogen atom.

6. The compound according to claim 1, wherein:
R4a and R5a represent an alkyl group or alternatively together with the nitrogen atom to which they are attached form a heterocycloalkyl selected from pyrrolidine or piperidine;
R4b and R5b represent an alkyl group or alternatively together with the nitrogen atom to which they are attached form a heterocycloalkyl selected from pyrrolidine or piperidine;
R₁ and R₂ represent independently a hydrogen atom, or a group selected from alkyl, benzyl optionally substituted by a halogen atom, benzodioxole, or phenyl optionally substituted by one or more identical or different groups selected from halo, cyano, nitro, alkyl, alkoxy, trifluoromethyl, phenyl, —(C═O)—R₁₄ or —(CH2)ₚ-NR₁₄—(C═O)—O—R₁₅ where p represents an integer between 1 and 2 inclusive.

7. The compound according to claim 1, wherein Y represents NR₁R₂, W represents —CR₆R₇—, R₁ represents a hydrogen atom and R₂ an aryl group optionally substituted by one or more identical or different groups selected from: halo, cyano, nitro, alkyl, alkoxy, haloalkyl, phenyl.

8. The compound according to claim 7, wherein R₂ represents an aryl group optionally substituted by one or more identical or different halo groups.

9. The compound according to claim 1, wherein W represents —CR₆R₇—, R₆ and R₇ represent respectively a hydrogen atom, n represents an integer selected from 1 and 2, and m represents an integer selected from 0, 1 and 2.

10. The compound according to claim 1, wherein the alkyl of the groups alkyl, alkoxy, haloalkyl, haloalkoxy, aminoalkyl, alkylaminoalkyl, dialkylaminoalkyl, aralkyl represents methyl, ethyl, propyl, isopropyl, butyl, isobutyl, or tert-butyl.

11. The compound according to claim 1, wherein the aryl of the groups aryl and aralkyl represents the phenyl group.

12. The compound according to claim 1, wherein the heterocycloalkyl represents a pyrrolidino, piperidino, morpholino, or azetidino.

13. The compound according to claim 12, wherein the heterocycloalkyl represents a pyrrolidino group.

14. The compound according to claim 1, wherein the cycloalkyl represents cyclopropyl, cyclobutyl, cyclopentyl, cyclohexyl or cycloheptyl.

15. The compound according to claim 1, wherein the heteroaryl represents the group pyridinyl, pyrrolyl, imidazolyl, triazolyl, tetrazolyl, thiazolyl, oxazolyl, thienyl, or furyl.

16. The compound according to claim 15, wherein the heteroaryl represents the group tetrazolyl or pyridinyl.

17. The compound according to claim 1, wherein said compound is:
3-({2-[(2,6-dipyrrolidin-1-ylpyrimidin-4-yl)amino]ethyl}amino)-4-methoxycyclobut-3-ene-1,2-dione;
3-({2-[(2,6-dipyrrolidin-1-ylpyrimidin-4-yl)amino]ethyl}amino)-4-piperidin-1-ylcyclobut-3-ene-1,2-dione;
3-(butylamino)-4-({2-[(2,6-dipyrrolidin-1-ylpyrimidin-4-yl)amino]ethyl}amino) cyclobut-3-ene-1,2-dione;
3-({2-[(2,6-dipyrrolidin-1-ylpyrimidin-4-yl)amino]ethyl}amino)-4-morpholin-4-ylcyclobut-3-ene-1,2-dione;
3-[(4-chlorobenzyl)amino]-4-({2-[(2,6-dipyrrolidin-1-ylpyrimidin-4-yl)amino]ethyl}amino)cyclobut-3-ene-1,2-dione;
3-({2-[{2-[(2,6-dipyrrolidin-1-ylpyrimidin-4-yl)amino]ethyl}(methyl)amino]ethyl}amino)-4-methoxycyclobut-3-ene-1,2-dione;
3-({2-[{2-[(2,6-dipyrrolidin-1-ylpyrimidin-4-yl)amino]ethyl}(methyl)amino]ethyl}amino)-4-hydroxycyclobut-3-ene-1,2-dione;
3-[(4-chlorophenyl)amino]-({2-[{2-[(2,6-dipyrrolidin-1-ylpyrimidin-4-yl)amino]ethyl}(methyl)amino]ethyl}amino)cyclobut-3-ene-1,2-dione;
3-[(4-chlorophenyl)amino]-4-({2-[(2,6-dipyrrolidin-1-ylpyrimidin-4-yl)amino]ethyl}amino)cyclobut-3-ene-1,2-dione;
3-{[4-chloro-3-(trifluoromethyl)phenyl]amino}-4-({2-[(2,6-dipyrrolidin-1-ylpyrimidin-4-yl)amino]ethyl}amino)cyclobut-3-ene-1,2-dione;
3-(biphenyl-4-ylamino)-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-{[3-(1H-tetrazol-5-yl)phenyl]amino}cyclobut-3-ene-1,2-dione;
N-(4-{[2-({2-[(2,6-dipyrrolidin-1-ylpyrimidin-4-yl)amino]ethyl}amino)-3,4-dioxocyclobut-1-en-1-yl]amino}phenyl)acetamide;
3-({2-[(2,6-dipyrrolidin-1-ylpyrimidin-4-yl)amino]ethyl}amino)-4-[(3,4,5-trimethoxyphenyl)amino]cyclobut-3-ene-1,2-dione;
3-[(3,5-difluorophenyl)amino]-4-({2-[(2,6-dipyrrolidin-1-ylpyrimidin-4-yl)amino]ethyl}amino)cyclobut-3-ene-1,2-dione;
3-[(2-chlorophenyl)amino]-4-({2-[(2,6-dipyrrolidin-1-ylpyrimidin-4-yl)amino]ethyl}amino)cyclobut-3-ene-1,2-dione;
3-[(3-chlorophenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-methoxyphenyl)amino]cyclobut-3-ene-1,2-dione;
3-[(4-acetylphenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(3-methoxyphenyl)amino]cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)methylcarbamate;
3-({2-[(2,6-dipyrrolidin-1-ylpyrimidin-4-yl)amino]ethyl}amino)-4-({4-[(methylamino)methyl]phenyl}amino)cyclobut-3-ene-1,2-dione;
3-[(4-cyclohexylphenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-propylphenyl)amino]cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)carbamate;
3-(1,3-benzodioxol-5-ylamino)-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-hydroxyphenyl)amino]cyclobut-3-ene-1,2-dione;
3-{[4-(aminomethyl)phenyl]amino}-4-({2-[(2,6-dipyrrolidin-1-ylpyrimidin-4-yl)amino]ethyl}amino)cyclobut-3-ene-1,2-dione;
tert-butyl[2-(4-{[2-({2-[(2,6-dipyrrolidin-1-ylpyrimidin-4-yl)amino]ethyl}amino)-3,4-dioxocyclobut-1-en-1-yl]amino}phenyl)ethyl]carbamate;
3-{[4-(2-aminoethyl)phenyl]amino}-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-fluorophenyl)amino]cyclobut-3-ene-1,2-dione;
3-({2-[(2,6-dipyrrolidin-1-ylpyrimidin-4-yl)amino]ethyl}amino)-4-{[4-(trifluoromethyl)phenyl]amino}cyclobut-3-ene-1,2-dione;
3-({2-[(2,6-dipyrrolidin-1-ylpyrimidin-4-yl)amino]ethyl}amino)-4-[(4-cyanophenyl)amino]cyclobut-3-ene-1,2-dione;
3-({2-[(2,6-dipyrrolidin-1-ylpyrimidin-4-yl)amino]ethyl}amino)-4-[(4-nitrophenyl)amino]cyclobut-3-ene-1,2-dione;
3-anilino-4-({2-[(2,6-dipyrrolidin-1-ylpyrimidin-4-yl)amino]ethyl}amino)cyclobut-3-ene-1,2-dione;
3-[(4-chloro-3-fluorophenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-(pyridin-3-ylamino)cyclobut-3-ene-1,2-dione; or
3-[(4-chlorophenyl)amino]-4-[(2-{[2-(diethylamino)-6-pyrrolidin-1-ylpyrimidin-4-yl]amino}ethyl)amino]cyclobut-3-ene-1,2-dione;
or a salt thereof.

18. The compound according to claim 1, wherein said compound is:
3-[(4-chlorobenzyl)amino]-4-({2-[(2,6-dipyrrolidin-1-ylpyrimidin-4-yl)amino]ethyl}amino)cyclobut-3-ene-1,2-dione;
3-[(4-chlorophenyl)amino]-4-({2-[(2,6-dipyrrolidin-1-ylpyrimidin-4-yl)amino]ethyl}amino) cyclobut-3-ene-1,2-dione;
3-(biphenyl-4-ylamino)-4-({2-[(2,6-dipyrrolidin-1-ylpyrimidin-4-yl)amino]ethyl}amino)cyclobut-3-ene-1,2-dione;
3-[(3,5-difluorophenyl)amino]-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-methoxyphenyl)amino]cyclobut-3-ene-1,2-dione;
3-[(4-acetylphenyl)amino]-4-({2-[(2,6-dipyrrolidin-1-ylpyrimidin-4-yl)amino]ethyl}amino)cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)methylcarbamate;
3-({2-[(2,6-dipyrrolidin-1-ylpyrimidin-4-yl)amino]ethyl}amino)-4-[(4-propylphenyl)amino]cyclobut-3-ene-1,2-dione;
tert-butyl(4-{[2-({2-[(2,6-dipyrrolidin-1-ylpyrimidin-4-yl)amino]ethyl}amino)-3,4-dioxocyclobut-1-en-1-yl]amino}benzyl)carbamate;
3-(1,3-benzodioxol-5-ylamino)-4-({2-[(2,6-dipyrrolidin-1-ylpyrimidin-4-yl)amino]ethyl}amino)cyclobut-3-ene-1,2-dione;
3-({2-[(2,6-dipyrrolidin-1-ylpyrimidin-4-yl)amino]ethyl}amino)-4-[(4-fluorophenyl)amino]cyclobut-3-ene-1,2-dione;
3-({2-[(2,6-dipyrrolidin-1-ylpyrimidin-4-yl)amino]ethyl}amino)-4-{[4-(trifluoromethyl)phenyl]amino}cyclobut-3-ene-1,2-dione;
3-({2-[(2,6-dipyrrolidin-1-ylpyrimidin-4-yl)amino]ethyl}amino)-4-[(4-cyanophenyl)amino]cyclobut-3-ene-1,2-dione;
3-({2-[(2,6-dipyrrolidin-1-ylpyrimidin-4-yl)amino]ethyl}amino)-4-[(4-nitrophenyl)amino]cyclobut-3-ene-1,2-dione;
3-[(4-chloro-3-fluorophenyl)amino]-4-({2-[(2,6-dipyrrolidin-1-ylpyrimidin-4-yl)amino]ethyl}amino)cyclobut-3-ene-1,2-dione; or
3-[(4-chlorophenyl)amino]-4-[(2-{[2-(diethylamino)-6-pyrrolidin-1-ylpyrimidin-4-yl]amino}ethyl)amino]cyclobut-3-ene-1,2-dione;
or a salt thereof.

19. The compound according to claim 18, wherein said compound is:
3-[(4-chlorophenyl)amino]-4-({2-[(2,6-dipyrrolidin-1-ylpyrimidin-4-yl)amino]ethyl}amino)cyclobut-3-ene-1,2-dione or a salt thereof.

20. An industrial compound that is a synthetic intermediate selected from the following compounds:
3-[(4-chlorophenyl)amino]-4-methoxycyclobut-3-ene-1,2-dione;
3-{[4-chloro-3-(trifluoromethyl)phenyl]amino}-4-methoxycyclobut-3-ene-1,2-dione;
3-(biphenyl-4-ylamino)-4-methoxycyclobut-3-ene-1,2-dione;
3-methoxy-4-{[4-(2H-tetrazol-5-yl)phenyl]amino}cyclobut-3-ene-1,2-dione;
N-{4-[(2-methoxy-3,4-dioxocyclobut-1-en-1-yl)amino]phenyl}acetamide;
3-methoxy-4-[(3,4,5-trimethoxyphenyl)amino]cyclobut-3-ene-1,2-dione;
3-[(3,5-difluorophenyl)amino]-4-methoxycyclobut-3-ene-1,2-dione;
3-[(2-chlorophenyl)amino]-4-methoxycyclobut-3-ene-1,2-dione;
3-[(3-chlorophenyl)amino]-4-methoxycyclobut-3-ene-1,2-dione;
3-methoxy-4-[(4-methoxyphenyl)amino]cyclobut-3-ene-1,2-dione;
3-[(4-acetylphenyl)amino]-4-methoxycyclobut-3-ene-1,2-dione;
tert-butyl{4-[(2-methoxy-3,4-dioxocyclobut-1-en-1-yl)amino]benzyl}methylcarbamate;
3-[(4-cyclohexylphenyl)amino]-4-methoxycyclobut-3-ene-1,2-dione;
3-methoxy-4-[(4-propylphenyl)amino]cyclobut-3-ene-1,2-dione;
tert-butyl {4-[(2-methoxy-3,4-dioxocyclobut-1-en-1-yl)amino]benzyl}carbamate;
3-(1,3-benzodioxol-5-ylamino)-4-methoxycyclobut-3-ene-1,2-dione;
[(4-hydroxyphenyl)amino]-4-methoxycyclobut-3-ene-1,2-dione;
tert-butyl(2-{4-[(2-methoxy-3,4-dioxocyclobut-1-en-1-yl)amino]phenyl}ethyl) carbamate;
3-methoxy-4-{[4-(trifluoromethyl)phenyl]amino}cyclobut-3-ene-1,2-dione;
4-[(2-methoxy-3,4-dioxocyclobut-1-en-1-yl)amino]benzonitrile;
3-methoxy-4-[(4-nitrophenyl)amino]cyclobut-3-ene-1,2-dione;
3-[(4-chloro-3-fluorophenyl)amino]-4-methoxycyclobut-3-ene-1,2-dione; or
3-methoxy-4-(pyridin-3-ylamino)cyclobut-3-ene-1,2-dione.

21. A process for preparing a compound of formula (I) according to claim 1 from compounds of formula (VI) 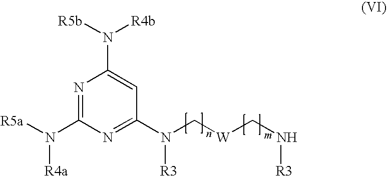
comprising:
a) reacting either the compound of formula (VI) with a compound of formula (VII) 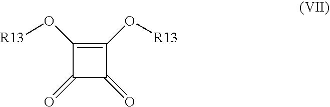
to obtain the compound of formula (I) where Y represents OR₁₃;
and reacting the resulting compound of formula (I) where Y represents OR₁₃ and R₁3 represents an alkyl group:
either with an inorganic acid to form the compound of formula (I) where Y represents OR₁₃ and R₁₃ represents a hydrogen atom;
or with an amine of formula HNR₁R₂ to form the compound of formula (I) where Y represents NR₁R₂;
b) or reacting the compound of formula (VI) with a compound of formula (IX) 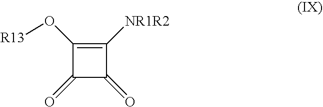
to obtain the compound of formula (I) where Y represents NR₁R₂.

22. A pharmaceutical composition comprising as an active substance the compound of formula (I) according to claim 1 or a pharmaceutically acceptable salt of the compound, and at least one pharmaceutically acceptable excipient.

23. A drug comprising the compound of formula (I) according to claim 1 or a pharmaceutically acceptable salt thereof.

24. A method of treating a disease or disorder comprising administering the compound of formula (I) according to claim 1 or a pharmaceutically acceptable salt of the compound to a patient in need thereof, wherein the disease or disorder is cancer, a cancerous proliferative disease, a noncancerous proliferative disease, a neurodegenerative disease, a parasitic disease, a viral infection, spontaneous alopecia, alopecia induced by exogenous products, radiation-induced alopecia, an autoimmune disease, graft rejection, an inflammatory disease or allergies.

25. A method for the treatment or prevention of cancer comprising administering the compound of formula (I) according to claim 1 or a pharmaceutically acceptable salt of the compound to a patient in need thereof.

26. The method of claim 25, wherein the cancer is cancer of the colon, rectum, stomach, lung, pancreas, kidney, testicle, breast, uterus, ovary, prostate, skin, bone, spinal cord, neck, tongue or head.

27. The method of claim 25, wherein the cancer is a sarcoma, carcinoma, fibroadenoma, neuroblastoma, leukaemia or melanoma.
